SEQUENCE LISTING (22 sequences)

SEQ ID NO: 1 - DNA, 23 bp, Artificial Sequence
  Oligonucleotide primer SSR 45F
ctcacgatccttaccttgaattg

SEQ ID NO: 2 - DNA, 23 bp, Artificial Sequence
  Oligonucleotide primer SSR 45R
atccactgtgtgcgtttctagtt

SEQ ID NO: 3 - DNA, 22 bp, Artificial Sequence
  Oligonucleotide primer C3-145F
acgggttgtttcacttacaggt

SEQ ID NO: 4 - DNA, 23 bp, Artificial Sequence
  Oligonucleotide primer C3-145R
tgtttaccaaactagccacccat

SEQ ID NO: 5 - DNA, 20 bp, Artificial Sequence
  Oligonucleotide primer b0060j21ssr1
gccatcctccactcctcatc

SEQ ID NO: 6 - DNA, 23 bp, Artificial Sequence
  Oligonucleotide primer b0060j21ssr2
tatgcaaactggacgaattaccc

SEQ ID NO: 7 - DNA, 23 bp, Artificial Sequence
  Oligonucleotide primer b0024J04ssr3
ataagcaagctcacacacacctc

SEQ ID NO: 8 - DNA, 23 bp, Artificial Sequence
  Oligonucleotide primer b0024J04ssr4
gctagctactctccaccactctg

SEQ ID NO: 9 - DNA, 20 bp, Artificial Sequence
  Oligonucleotide primer A87o09ssr1
gatgtcctctcccaccttgc

SEQ ID NO: 10 - DNA, 23 bp, Artificial Sequence
  Oligonucleotide primer A87o09ssr2
agggtgtacagtcagcacctctc

SEQ ID NO: 11 - DNA, 13732 bp, Oryza sativa
  misc_feature (1)..(13732): rice genomic GO
ggacccaggtcaggtccggttgctagttcttttttcttttttttcccattgttttaaacattgaatattgatttcactccatccccgaatcatccccagatcgtgaaatcccaaatcgatcatcacagatcagagaacaaaaggatatcttcaaatcctcaaatcgatcgtctccaaatacatcacaaattctaaaaaaaatacatcacaagttctaagaaaatcatcacaaatacatcacatatttacatcacacacaaatcaaatacatctaagatccgaatcataaattctcaaaaaaaaaatccgccatccaccaccctccgctggcctcgctcgatctgctctcgtcgatagaggaggaataggaaggagaggataagagagaggaggaataggaaggagaggataaggataaaggagaggaatgggaagatgagcaatttgggaggataagtcctagggattatatactgtgtatggtttttttacccactgggactaaagattaattttcaatcctaaaaatgcttggccccctgacagtctcggtcagaaataggaaggagagtgtaagagagaggaggaataggatcacctaggactaaagatcacctagggccgacagagttttaaaccgagataaaaaaaatcatatttagttccggtttctattcaaaccgggactattatgttttttgtccacccaacaaagatggtttatccagtagtgatgactaataggggaatgactagaatggatgcggagggagaatgattgttgccgagcaaccaaacaaagccttctggatagaagccatgttgctgttgagtggatgggcacataaccgaggaatcggtgccagagagcccttgagtccatgttgttgtcgagtgaatggcacatgagccgatgaatcagtcatagcccttgagtcggctacccgtgcgattcctctcttgacttcgcgtgtgggttctcctgtgcgaaaaggctaatggtcagcccatttcctttttggcccactcggttttggcctttccttttttttactaggccttgtttaattccaaaattttttttgtcaaaaacgtcacatcaaatatttaacacatgtatagggcattaaatgtgggaaaaaatcaattgcacaattcgtatgtaaattgtgagatgaatcttttaagcctaattacaccatgatttgacaatgtgatgctacagtaaatatttgccaaggacggattaattaggcttaataaattcgcctcgcagtttacatgtgaaatctgtaatttgttttgctattagtccacgtttaatacttcaaatatgtgtccatatacttcaaaaactttacacccaaagaactaaacacaccctaatcattttataggcttatggtcacttagctttctttattcggcagatcgagtataaattgagcctctttgttttcctactaggccatttcttatcatcccacacgtattttttgccttgtctatttttctaatttattttaaaaaattgattgaagttatatgtacatgttgtacgtacaaagttatatatgtatatgtaaaaatataaagtttatatgtataattttttattgggctaatgtgctactgggccagaaatcctcttgttggccattttaaaaaaaagagaaaaggaggcgtagagcaccggaaaagctaatacgcgcgagccggcggtgcgcgtttcgggcccagaaggcccagcgtcgctgtgctggcgcgcgtgcgtctcctccactttttttttaccttttttttttccttttttcccacttttttcggttttttatctagcatgttttgagttcgaaagttttaaagttttgagttgaaaattttcaaatctgaacttgaaagtttttaaatcccgacttgaaagttttcaaatctcaagttgaaagttttcaaatctcgatgaaaagtttcaaaattttcaaatcttagatttgaaagttttcaaaatctcaacttgaaagttttcaaatctcgagttgaaagttttcaaatctcaaattgaaagtttttaaaattttcaaatctggacttgaaagttttcgaatctgagtagaaagttttcaaatttcaaggtgaaagttttcaaatctgagttgaaagtttttaaaatttgaaacttaaatcgaaagtttttagatctaacttaaaaaaaatttcaatatgttagtaaaaaaatctctagaatctttcctaattactatcattagtgttaagtacattaactaatatagttacttaagactaaatggagggaggaatactcgctgctgacgcgcacacgccaactagcaacctccgtagatcacacccgtattggttttgtccatgccttattttttcgtttccaattgtgcgtcccttgtgcgtgattgaccgtggattagaccacccctctcctgtgttgttcttgggccaaagctggcgtggcacccatgttatcaacctgggtccggaatgcaaatatgataaatgtatatggtgattaaataaaactatagggttaccttaaatttattgtaaaactttagtaactaatactaataaaagtgatgagtgaaattaactcaattaaaaaagcggcatatgctctgttgctcaccttagcactggacactggtaggtgtgaagcaaacgtcagtggtaaagttggtatcagttggactccgttgtttatacgtcggtgctcgtccttttctgttcttcttcaatctgatttgcttaggtgtggaaccaacaccaaacctttaaatctactgctctactttttttttaacaactgtactatttgctggcatccgttaaaagagcaaactggaattgtttacacgtccctctagtctcatggttccagttctagtatactagagaatctgaacactactccctccgtccaaaaaaaaaaaaaggcaaacgttgggtttccgtgtctaactttgactgttcgtcttatatgaaatttttttataattcgtatttttattgttgttagataataaaacatgattaatattttatgcgtgacttatctttttaattttttttataattttttcaactaagacggacagtcaaacgttgaatacggaaaccagggtttatctttttttgggacggatggagtacgttataacccatgtggtcaataaaattgataataatctcaaaggatatccaaatcctatatactttcatagagttgaagaaaaagtttatacatgtaccataacccctcagcgtgatgtcttcgaatgatcaatccatagtctaatgtgtatagccttttttctatgattgatcaaggccacaaatctctttcatttgcaccttatttccatctctgccaaacaatattcccgtggcaaatgcccggaattttatccaataatactataaaaagtaaagagcacttgccacaattccactatccatatggcaaattgtttggagtagccgccttcggatgagatcaaggatacactacctacattcccgaacaaaaataaaaggaacaatatactccatggtgaactgctagatagccaccaggaatccacgatgcgacagcctatccatccatcgccaaccaaagcgcccgatggagcatcctctcactctcccgatcacgcaccagaatttccacgcggctgcaaacatgcaactctccggacgcgatgcgccccatcgctgtccaccgctcgatctatcaatctttcggcggcacacgccggaaaggcgagctgccggcgacggccagggccccctcgacacggccaccaccacaaccgaacaccctgttccgagccggtgaaaaaaaacgccaaaaaaccccacgtagcgcactagcgtctgtagctgcgccactgccaccgccaccgccaccgctggccaaagccaatccaagcttcccgttccgcccccgtaaaaaaaggcgttccaagcggcggcgtacttatcgaaatggagcttgggcccccctcctcaacacggtgcgaatgcccgcattaactagagcgctgcgctgtgcactgctcgctgcacgggtggcactcgcacgcgagaaagcacgccgcgcattcgcctctcacggcgaaatgctgtcgcagcccgccgcggcgctggctcgcttgccccccgcgtcggtgcgcgtcctcgtcgggttcggggcggcgctgctcgtctcgctcgtcgtcctccaccgccgccccgccgcgaggggggcggcgggcggcggcggccccgcggagtacgacccggccgcgcggttcctggccctgtccgagggggcgaacgcgaccatcgccgccgacctgcgggcgctcaccgcggggccgcacctcgcggggacgggcgcggcggccggcgccgccgcgcgggtgctctcggggttccgcgccgcggggctccgaaccctaacgcgggagtacacgccgctgctctcgtacccggggcacgcctccctcgcgctgctccgcgccgaccggaccctcctcgcgcacctgtccctcgacgagccggcggacgtcgggcgccgcctcgtgcggccgtaccacgcgtacgccccgtccggcggggccgtcgcggaggcggtgttcgtcaacctcggccgcgaggaggactacctcacgctggagcggctcggggtgagcgtccgcggccgcgtcgcggtggcgatccgaggaggaggctaccgcggcggggtggtgcgccgcgccgcggaaaggagcgccgctgccgttctcattgccggccacgcggacggcggggtcgagaggggcacggtaatcctcggaggccccggcgacccgctcacccccgggtgggccgccacggccggggcggagcgcctggatttcgaccacgaggatgttaagcggcggtttccggccatcccatctatgccggtgtcgggcaagacggcttcggctatcatacggacactaggtggcccggctttgccggcggattggcagaccggagttggactcccggtggatgtcggcggcgttggacctggtcccactctggtcaacttcacatatcaggtataaaatacaaatcgcctggaggattgtctatctatgattactcgcttagagacaaggacgctgggtacgtttgcagggaaacaggaatcaaagccatggaccgctcctttgacctatcttgactgggttttccagtgataggctgttgccattttgttttcaacttaagagagacgtctgtgctgaatggggaaagagtcatcgtagtagtccatgttgtaaactgccgtgttgagattctggataagctcctaaattgtttgagctgtaaaagatgtcactacatttgcagtttgattaggaagcacagtaaccatttccttgtccgcttgactattttggtttcagtggacagtggtcctagcacctgttgttttattagcccttttttatatctgtggtagtgtgtacaaaactacaaatagcattatgcttgatgttgagcgtattgaccagcctttgctcctttgtagtgcatattggttaaacttagttagattattattgttacagatttatacagaatgagaaatattattgtgggcatactgagctatgagcaccaaatcattgagcacttgtgacgtaagttttgcaagtggtatgcttttatggctccctttgttatgttgctgtaggcctatttgtcattattctgtgttgtttctatatgctggatctgttgtcatgtctctcaattggatgagctatatatattggacaaataaagcattgcataatagctgtccgtgcttgtcctccaggcaagttagtgtttcaggatgcatcttttcttttgcacagttttgcatatttgtataactggcagatgagactatgttttagagttaagtacaatcaagaccataatggttttctcttatatcttatttaaggaggacaggaagatgggcatgatacaggatatttttgctattattaaagggtacgaagaacctgatcgttacgttatccttggtaaccaccgagatgcatggacctatggagcagttgaccctaacagcgggacatctgcgcttcttgatattgctcggcgtcttggaataatgctgcagtcaggctggacaccacggaggaccatcattctttgtagttgggatgccgaagagtttggaatggtgagttcatattaaagaaaattgagaatatgttgctgaacaccatgtgaaaaaggaaaccatgcagatgcccccacaccctaacattttcaaagtaacatatatggcatttgcattttatgaaaggaaccccacaaaaaaatcatatatagttggcatgttcgtagctatccttttgaatgaacttttctggttgatttgattcatctgtcacagttctcaaagatctcatttatttgtgtcccgtttatttcttttctacattttgaaggttcttttttccagatcggatcaactgaatgggtcgaagagaacctcgaagatctgcaatcgaaagctgtagcttacttaaatgttgattgtgcggtgcaaggaattgggctttttgctggttctaccccccagttggacaacctcttggtcgatgttacaagacaggtagagattaatgtgtttccattgcatttttctatgagcttgggttttcatttagaaacctcagtccactcagctatttataccaaattttagaattgttctgaagtgaagttatgtttattcattcagaactttttgcttctaggtaaaagatccagatgttgaaggaaaaacagtccatgatacatggaataaaatgaccggaggcatcaatgtaagatctcgaaggtttaaacatgcttttctatatacatattgagatgtactcaatccaggttgtcactagctgttctaaactggtttaaagtcaaacatcacttttataatagccatctagtggtcacactttgtacactacagggttggtaactttgaattatctatgccgacaactaatcttctgcatagtgagttactgagttggttgttcttttgttttgagtttttagtagggtaaggtatgcagttctacattgggtgcagttgctccaagtgcagggggcaggcccatatagatctcagttcctagagtgagacatcccccacacaagatatacttacgagatgtgctaatctggtgtcgtcgaatctttgttgtagtcgacagtctacatgacaactatacaaaaatgaaagtgaaaattactacttgctatgcactgatcttatacttctgacgtgagcaattccttctttctttttttcaaatccttacaaaacaggtaatataaatacatgaaattgttatttagataaagatatatcgatattggtttagtagaacgcatcagtggcacaaagaacgaaccaaaaataatagtaataaaagataacgataacttcatgtttcataacttcaccttatataggcatctagccctcaccaccttcatgggctaacgcaggcccctggctgtggcccaggtggcctggggcctgaaagtgaaaacctattcatagaaactagccaaatacaggctgggccaacccggaattaagcccctgaccaccttaaagctagttgggggtggcacctccaagataaccctctaattattaggactatcacatagtatgaattaactaattacttggtatctggctggttgatctttcaaaattgtttcgctgaaattctttgtcagaatttttgaaaaaccttctgaatgaaatatggtcaacaatttatccatcagcaatgattagttggcatgaacagctaattttgtaacatctggaatgactcccactcatttttgtgtcaagcccacgtcataattactccaagcactccaaatgatggcttccataaataccatggaaagctccaaaccttacaaaatgtcatttgctttccacgacaatcagatttaccatttaagggcgatacctttatgtagaaaaaatttccgttctgttgaagttgacatggctgtaccagttgaaggtgcatgtgaaagtcagctgtatcggttgcagagaataatcgctactcggttgtctccatcaagttgagcatgcaaggtctggatcccacatgcaatcttagtgctagtcaaaattcacactttctacatagaattgtggaataagataataaccaaattgtgtcgatggatattccagttccaataaattaggctacaaactacctgtacctggttatacattttttgtgttgcatactgttacatattaagaatatgcagtccgggtttatcactggactaactttggtcaaggagcatcaaggtgtgatgcatcacccagttgctcactcaacaactgtctcaagtcccactgtccctttgaaactcttgtttccgaccatgcaattagatgccttggcccattgcttctcaaggcctcaaccacatactaagtgctatacaacttggtccctaccatgttaacccagccaggacgtctgaaataattgtcctaactggcgcggggaaaacctcacctatatcaggcacatgtatgtcccactttcccatgcctatttaccagcgggtgcacaaatacaatgtagattgatgaggtacctatgatgtgtatgattaaaatcatgtgtatgtgtgaagtgtagaactgtaaattaggcaactaatttccacagccagttcacgtgtccatgacactccatctgtcttgttccctgtcttcaatgaacaaactgcttattgtggtcctccttgctgtagtaccttccatcacctcacttgtgtgcctgaagggtgagtttctggcgcaggaggccaagcattctgccagacgcccgaggagccattccatctacttcactctgattttgtctaaacttttttattgtgctgtaaattaattactatcgcttgctaccgtaagtgaaaaaaaattctggttatgatcttttgagcgtctaactcatatatatgttctgattatttactgacaatgaattcgcagatagaaagacttgccagaactgattctgactttgctccatttctacatcatgctgggatcccctgtatggatttatactatggaaaaggtattttaagcatgcaatcacgtctttctgtttattattgttaaattaacttataactttccttttccttctccaatatagcatgtaatatacggagaaaaagacttgctttttttgctcaaaaaataaactgtttttttctcaaaagagagaacagtatatgttttctaagcagaggacattaatgcaaccccattttttaggatcgttactgcttgctcattttttaaaatattttgttgagctatgtgatttaattttgtacatctctcaatcgtttcctggttccaagataaagttagaagagacaaaatatttccccatcaccccactttctagagagaatgcaggacatcatctaactggtctaatacgcagaatttcctggatatcatacggctcttgactcgtatcattggatggaaaagcatggtgatccactgtttcttcgtcacgtggctagtaagtagcagtaatgatcttttaaaatgattctgaaagggagaaaatgtgcaataccttacaaactacaattttgcttgttcaaattactgttaacttgaatgcactggtacaagaccagtcaattattataaaaattctatggactatcccatatagaactagacataaggtcgcagctgagtttggtatttttgtgctttaaaatatgttgtacacatctcttataatactcatcatgctcatttactttggctcaacagttgtggagatttggggactactggcccttcgattagcagatgaccctgtgctgccttttgattatcaaacatacgcttcccagttgcaggtacatcgattctttcagaattagcacaagcatttaacattttcatatgattggcatttatcatcctgaaggactgtacgtgtatgtttatgtagcatattcaatttttgcttatgcaatgggagcttgacccaaaaacagggttttgctcaaaacaacattgtaattgacaattttgaatgcacatatgctcaatctagttgaactgaactttagaacacaaatataagtcacgcatgatgaccaacttcatgggttcatagatcatattagagtaggggtatatttccttttcccacagcatcatggtctcatcttctgatgtacattttgaacttgcaatgtcgcaggagcatgcaaatgcattctctagtatgatggaaaatagcaaatgggttcatttgctgaatagatccattgaagatctttctgatgcaggcttagaatttctgaaagaggcaaaggtatgttcaagaatctttttttcttttgctttatattctgttttgtctttctgtttccatgtaattaggtagtctgtagtccattatagttgcggttggctgaagatattttcttctttagctctttcactctgcacactgcatatcacttctgtatgtacaaccagtttgaaacggcagtatgtatatttggatatccaattttatttagacatttgccttttgcagttttgctgcacatacaggcaataactttaatttcttagaacttagtaatccttttggtgcctgacatcacattattgatgctttctcttaaaaattattatatcagtacagttatttttccattcccatgatggatgccatttattttgccacttggatctcatgttttaaatattttgtaccaatctatattgccgatgataaccttattttcttttgttgatttggaacattctagaaattacaggaccaaaatatatccgatgggtactctctgatgaggcggcgattgttgaacgaccgtcttctacttgctgaaagaagctttctgcaagctgatggacttcagggaagaggatggttcaagcatctggtgagccttaagtgggtcagccatgaacccacttataggtcaaaataagcaggtctgcggcttattttggcctataagtgggtttcgcgggttagccacttacactcttaagcctcccgctattcataagtcataagtcataaccaaatttagaaaaaaaaaatgttgtgcgtgaccacgcttttatatttcttgctagatgtactcgcctccggaggactacgaaagcaagctctcgtttttccctggggtggcggacgccatctcccggtcgagcaatcgaagcgcgaaagaacagcaggcggcggtgcggcatgaggtgcggaaaatctctagggcaatccaaagagcagctgatgttcttagaggcgaattcagtaataggaacgaatctttatactctagcctttctgtggctccataaaaactgtgtataaataaaatcgattggggtttgagaaatgaaatcttgttcctgtgcgaaggtggttttatattgcgtttcttgacactactctttttagagggcattatgcttaattaaaacaagaagccaggattgcaatctgaacagcaacaacagaaattaggaagtctgatggaagtctaagctaagtttgaagaaactaggatcgcagctattatttgcaccggcaaaccaactattcctagcacctcaatttagtctttgttttttcataacagaaaagaaaaggagaccaatagcactttggaaaaccagtgcatgttaaaccgcccaccaactaccagggtttttaaaccgccgagcccggagttaaccgcgtagtaaggcaaaccctgccaaccacacctcaaaccccccttaaaaaaaacctagagctcatattagctagtactccctccatcctaaaatatttgacgccgttgactttttaaaaaatgtttgatcgttcgtcttattaaaaaattaattaattattaattcttttcctatcatttgatttattgttaaatatacttttatgtatacatatagttttacatatttcacaaaagtttttgaataagacaaacggtcaaacatgtttaaaaaaatcaacggcgtcaaacatttagtgaaagagggagggagtactttactgtacaccacgttcatattgctgtgagctcaaattaaatggaataaatataataacgaaaatcctaccagctacccacaaacggtagccggcaggaacagcccaaacctgcctgaaatggaaccgcgaaaaaaacggaaacgtgggtagcgcgcgcgcgcgctcgcgctcgcgcatctgctcatgccgttgcttagtattctttctcgtgcaattattcaactaatcacaaataattatagcgttaattttttcccatgcaaaatactccaactagccgttgccacacttccatgtagagacgaatccaccgtctcctctcctctcgtacgcacatacatagagccggccatggatttgccaaatcccaatctccccatgcaaaatcttgccaagatccacgagatcctgaaccaaatctccatctcccctccgtgagatctcccggtagatcatgtcccccggtgaggaggacgtggaagccagcagcgacagcggcggctccgcgacaaggcgttgcgccgcgtgcaagttcctgcgcaggcggtgctcccgcgactgcgtcctcgcgccccacttcccggcttccgatccccacagatacgcctgcgttcagcgtgtcttcggcgccggcaacaccgccagaatgctccaggtgatcactgatcactactactagtagctagctataccacacatactaccacactcatatttctaaacggaaattacacgagctggtatgttttttcttttttttgagcgtggtatgttttttcactaaaataattttaccattattaataaaaaaaataccaatgtaaatctagcagtatttcgaggtatcaaaatttctcttttcactatcacttggtagtgtgcgtgcgttggtttacgtcttctatgcaattgaaaaaaaaacatatggggctatattggtgtcactagctcactcactgtacaccttattcagttagaagttggcacgtgatcgaagttgttacttgttagtgataaaattgttgatgtgtagtgtaattggggaaagtgaattcatccctcgagaggatatcccgtcgtttcacatatgtcacataaatagttattaaaaaatatgaaaaaaatttagtaacatagattaatgtgaaatatatcactccacaaacatacaagtttaaattcaacttttacaagttgtaacaaaaacaacaaatatagttgtaaatgtgcgataactattttcaatttaatttgttatttttgttgcaacttatagaaattgaatttggttttgcatatttgtggagtgatatattttatattaatctatattgtcaatttttttttaaattatttaataactatttagatgacatgcaaacatgtcctctcgagagatgaaaatccacgtcctaataattgggctgcaaattggaacagagctatactccatatgttaatttcgcagtgatgcgcgtctcaattctatcgttgtattgcgtgcagcaact

SEQ ID NO: 12 - protein, 716 aa, Oryza sativa
  MISC_FEATURE (1)..(716): from genomic sequence
MLSQPAAALARLPPASVRVLVGFGAALLVSLVVLHRRPAARGAAGGGGPAEYDPAARFLALSEGANATIAADLRALTAGPHLAGTGAAAGAAARVLSGFRAAGLRTLTREYTPLLSYPGHASLALLRADRTLLAHLSLDEPADVGRRLVRPYHAYAPSGGAVAEAVFVNLGREEDYLTLERLGVSVRGRVAVAIRGGGYRGGVVRRAAERSAAAVLIAGHADGGVERGTVILGGPGDPLTPGWAATAGAERLDFDHEDVKRRFPAIPSMPVSGKTASAIIRTLGGPALPADWQTGVGLPVDVGGVGPGPTLVNFTYQEDRKMGMIQDIFAIIKGYEEPDRYVILGNHRDAWTYGAVDPNSGTSALLDIARRLGIMLQSGWTPRRTIILCSWDAEEFGMIGSTEWVEENLEDLQSKAVAYLNVDCAVQGIGLFAGSTPQLDNLLVDVTRQVKDPDVEGKTVHDTWNKMTGGINIERLARTDSDFAPFLHHAGIPCMDLYYGKEFPGYHTALDSYHWMEKHGDPLFLRHVAIVEIWGLLALRLADDPVLPFDYQTYASQLQEHANAFSSMMENSKWVHLLNRSIEDLSDAGLEFLKEAKKLQDQNISDGYSLMRRRLLNDRLLLAERSFLQADGLQGRGWFKHLMYSPPEDYESKLSFFPGVADAISRSSNRSAKEQQAAVRHEVRKISRAIQRAADVLRGEFSNRNESLYSSLSVAP

SEQ ID NO: 13 - DNA, 2273 bp, oryza sativa
  misc_feature (1)..(2273): cDNA insert in rls6.pk0079.c3
gttctaacattaactagagcgctgcgctgtgcactgctcgctgcacgggtggcactcgcacgcgagaaagcacgccgcgcattcgcctctcacggcgaaatgctgtcgcagcccgccgcggcgctggctcgcttgccccccgcgtcggtgcgcgtcctcgtcgggttcggggcggcgctgctcgtctcgctcgtcgtcctccaccgccgccccgccgcgaggggggcggcgggcggcggcggccccgcggagtacgacccggccgcgcggttcctggccctgtccgagggggcgaacgcgaccatcgccgccgacctgcgggcgctcaccgcggggccgcacctcgcggggacgggcgcggcggccggcgccgccgcgcgggtgctctcggggttccgcgccgcggggctccgaaccctaacgcgggagtacacgccgctgctctcgtacccggggcacgcctccctcgcgctgctccgcgccgaccggaccctcctcgcgcacctgtccctcgacgagccggcggacgtcgggcgccgcctcgtgcggccgtaccacgcgtacgccccgtccggcggggccgtcgcggaggcggtgttcgtcaacctcggccgcgaggaggactacctcacgctggagcggctcggggtgagcgtccgcggccgcgtcgcggtggcgatccgaggaggaggctaccgcggcggggtggtgcgccgcgccgcggaaaggagcgccgctgccgttctcattgccggccacgcggacggcggggtcgagaggggcacggtaatcctcggaggccccggcgacccgctcacccccgggtgggccgccacggccggggcggagcgcctggatttcgaccacgaggatgttaagcggcggtttccggccatcccatctatgccggtgtcgggcaagacggcttcggctatcatacggacactaggtggcccggctttgccggcggattggcagaccggagttggactcccggtggatgtcggcggcgttggacctggtcccactctggtcaacttcacatatcaggaggacaggaagatgggcatgatacaggatatttttgctattattaaagggtacgaagaacctgatcgttacgttatccttggtaaccaccgagatgcatggacctatggagcagttgaccctaacagcgggacatctgcgcttcttgatattgctcggcgtcttggaataatgctgcagtcaggctggacaccacggaggaccatcattctttgtagttgggatgccgaagagtttggaatgatcggatcaactgaatgggtcgaagagaacctcgaagatctgcaatcgaaagctgtagcttacttaaatgttgattgtgcggtgcaaggaattgggctttttgctggttctaccccccagttggacaacctcttggtcgatgttacaagacaggtaaaagatccagatgttgaaggaaaaacagtccatgatacatggaataaaatgaccggaggcatcaatatagaaagacttgccagaactgattctgactttgctccatttctacatcatgctgggatcccctgtatggatttatactatggaaaagaatttcctggatatcatacggctcttgactcgtatcattggatggaaaagcatggtgatccactgtttcttcgtcacgtggctattgtggagatttggggactactggcccttcgattagcagatgaccctgtgctgccttttgattatcaaacatacgcttcccagttgcaggagcatgcaaatgcattctctagtatgatggaaaatagcaaatgggttcatttgctgaatagatccattgaagatctttctgatgcaggcttagaatttctgaaagaggcaaagaaattacaggaccaaaatatatccgatgggtactctctgatgaggcggcgattgttgaacgaccgtcttctacttgctgaaagaagctttctgcaagctgatggacttcagggaagaggatggttcaagcatctgatgtactcgcctccggaggactacgaaagcaagctctcgtttttccctggggtggcggacgccatctcccggtcgagcaatcgaagcgcgaaagaacagcaggcggcggtgcggcatgaggtgcggaaaatctctagggcaatccaaagagcagctgatgttcttagaggcgaattcagtaataggaacgaatctttatactctagcctttctgtggctccataaaaactgtgtataaataaaatcga

SEQ ID NO: 14 - protein, 716 aa, Oryza sativa; identical to SEQ ID NO: 12
  MISC_FEATURE (1)..(716): translation of nucleotides (100)..(2250) in rls6.pk0079.c3

SEQ ID NO: 15 - DNA, 2260 bp, Zea mays
  misc_feature (1)..(2260): from BACM2.pk146.m06 (1)..(567) and csc1c.pk003.k10 (568)..(2260)
atgccgcactccgtcctggcccgtctgccccccggttccgtccggctcgtagctgccttcgggctgctgctcctcgtctcgctgctcgttctccaccgccgccccgggaggccccatgtggcggcggccgccgccagcgaccgactcactgatccctcccggtcccggctgttcctgtcgcagtcccccggggccaacgcgagcatcgcggctgacctccgcgcgctcaccgcggggccgcacctcgcggggacgcctgcgtcggcgggtgcagccgcgcacgtgctcgcccgcctccgcgccgcgggactccaaaccctaacgcgcgagtacgagccgctgctctcgtaccctgggcacgcctccctcgcgctgctccggcccgacgggtccctgctcgcgcgcctgtcgctcgaggagcccgcggacgaggggcgccgcgtcgtgcctccgtaccacgcgtacgcgccgtccggcggggccgtcgcggaggcggtgttcgtcaacctcggccgcgaggaggactacgtcgtgctcgagaggcttggagtgggcgtgcgcggccgcgtcgcggtggcgcgccgcgggggtgggtaccgcggcggggttgtggcgcgcgctgcggacaagggagccgtcgccgtgctcatcgcgggcaacgcggacggcggcgtcgagagaggagtcgttcttctcggcggccccggcgatccgctcacccccgggtgggccgccaccagcggtgcggagcgtttgaagttcgacgataaggcagtcaagcagcggtttccgagtatcccctccatgccggtgtcggccaagacggcagcagcgatcattcgaagcctgggcggtccggccataccggcggaatggaaggacggcctcggggtggataccggtggtcttggacctggccccacgctggtcaacttcacgtatcaggaggacaggaagttttataagatacgagacatttttggtatcataaaagggcaagaggaacctgaccgatatgtgatactcggtaaccacagagatgcatggacctatggagcagttgatcctaacagcgggacagctgcacttctagacattgctcggcgtcttggaataatgctgcaatcaggatggaaaccacggaggtccatcattctttgtagttgggacggtgaagaatttgggatgattggatcaactgaatgggttgaagacaaccttgaagatctgcattccaaagctgtagcttacttgaatgttgattgtgctgtgcaaggtgtgggattttttgctggttctactccccagttggacaaactcttggttgatattacaagacaggtaagggatcctgatgtcacgggaaagatggttcatgatacatggaatgaaatgagtggcggcatcaaaatagaaaggcttgccagaactgattccgattttgctccatttctacatcatgctggaattccatctgtagacttgtactacggagaagaatttcctggttaccatactgctcttgacacttataattggatggaaaagcatggcgatccattctttcttcgtcatttggctatcacagaaatttggggactattagctcttcgattggccaatgatcctgtgttaccttttgattatcaagcttacacttcgcagttacaggagcatataaaaacattgtctgccctgacgagcaatggccatgcagtaaatttgatgaatgggtgcgtcaatgatctttctggtgcagctatggaagttctgaaggagatgaagaaactacagcagatggatttatatgatgaacacgccaggatgagaaggcgattgttgaacgatcgcctcctacttgctgaaagaagcttcctgcaacctgaagggctccaaggaagaggatggtttaagcatctgttgtactcgcctccggaggactacgaaagcaagctgtcgttcttccctgggatcgctgacgccatctcacggtccgccaatctgagtgacaaggaacaggaggtggcaatgcagcatgaagtgtggaaagtctgccgtgcgattcaaagggctgcgagtgttcttagaggtgaattcagtgagcaaaaaccgacgaattttagttctttggtgaccccatgagatgtgtttgtgcaaacaaataacaaacaatggttatttagtatctgcgcgaaatggaagtgtgatgaagaatgtgaaaaaaaaaaaaaaaaaaaaaaaaaaaaaaaaaaaaaaaaaa

SEQ ID NO: 16 - protein, 713 aa, Zea mays
  MISC_FEATURE (1)..(713): translation of nucleotides (1)..(2139) of SEQ ID NO15
MPHSVLARLPPGSVRLVAAFGLLLLVSLLVLHRRPGRPHVAAAAASDRLTDPSRSRLFLSQSPGANASIAADLRALTAGPHLAGTPASAGAAAHVLARLRAAGLQTLTREYEPLLSYPGHASLALLRPDGSLLARLSLEEPADEGRRVVPPYHAYAPSGGAVAEAVFVNLGREEDYVVLERLGVGVRGRVAVARRGGGYRGGVVARAADKGAVAVLIAGNADGGVERGVVLLGGPGDPLTPGWAATSGAERLKFDDKAVKQRFPSIPSMPVSAKTAAAIIRSLGGPAIPAEWKDGLGVDTGGLGPGPTLVNFTYQEDRKFYKIRDIFGIIKGQEEPDRYVILGNHRDAWTYGAVDPNSGTAALLDIARRLGIMLQSGWKPRRSIILCSWDGEEFGMIGSTEWVEDNLEDLHSKAVAYLNVDCAVQGVGFFAGSTPQLDKLLVDITRQVRDPDVTGKMVHDTWNEMSGGIKIERLARTDSDFAPFLHHAGIPSVDLYYGEDFPGYHTALDTYNWMEKHGDPFFLRHLAITEIWGLLALRLANDPVLPFDYQAYTSQLQEHIKTLSALTSDGHAVNLMNGCVNDLSGAAMEVLKEMKKLQQMDLYDEHTRMRRRLLNDRLLLAERSFLQPEGLQGRGWFKHLLYSPPEDYESKLSFFPGIADAISRSANLSDKEQEVAMQHEVWKVCRAIQRAASVLRGEFSEQKPTNFSSLVTP

SEQ ID NO: 17 - DNA, 10288 bp, Zea mays
  misc_feature (1)..(10288): BACM2.pk146.m06
tcttttgaggaatacacctatatgagtcagactgctggtcacagtctatgggacttgggtaatccctaaatactcatgaaaggcactgaagtgtgacttatatgcttctaaacagcgggaatacccttttgcagccaagtatcagcaaaggatttgagtgaatcttatttcgcacaaaactgtcaattcaaggcatagtccattgttcagttgtgaatgagtgaaactcttattctagatggatgttcaacttaacagtctccatcgaaacactggtatatcaaaggattgtgattctgatacttcatcattacaaaccctagagtttggtggggattgttggatcttttatgggcttggcccatttattgaataaactctaaggtgtgtaatggtggataataccaatagtaccacattggaagtccaagggtcttttgccttgacttatatggtgggatttattccacttaacttgagaagtcaagaaatggacaagggcgtgccacacgcgcgcgcgtcgccgccgccgccggccgggcgtggcgtggcgtggcgtggcgtggcgaggcaggcaggcaggcaggcaggcaggcaggcgagcgggcgtggtgtggttgtgttaatttttagcactcactaaccgcgtacgtcagccgagtgaccctgtctcttcgtcagccagccgagtgacccttctccttcagctgccgactcatggcgtgactcggcgagagctggccgactcatggcataactcggctagagctggccgactcttggcgtgactcggcgacctttctccttcagcttccctctgcgagaggataaatagaggagcacccctgtcacttggtacacgcgagaacactttcagaatcacagttccagccgccgagtgagggtatttccatctcgtgactctgcgcgcacagagaagcgagagggcaggtgcctccgaagccggtgccgttcgagaccttgcacgggggatcggcaattaggtttttggggagcgtctacgcgactgcccaaaacatctccaacatgacaaaagaagatgatcaaggatctggatcatcagagcgcatcggagggtatgatcgttttatttacttactgttatatgttctggatattatatatgtttcatgtattcatctatgctgtttcatatgtaggaatgattttatcttatctgttctgtcttattgtaatatgatagatctgtctgttttaattttacagtcatgcagtttgtgtttctatctgtttattttcaattgttcagtcagtttacatgtttatcgtatttatatgatttatataattcatatgctttatgttctcattatccatattgttatggatatatttgagaaagtgatttctatgattaattatattttacatgtcatcatcataatgttaatttatggaattaaaatgatatggaaaatgcctataattctaacaaagtcactaaaaatttagagcatgtttgattcgttatctaattatcatagtttacctatttttctaaagttagttttttatttaactgactaacctatgtgatcatgtaaaatactgttttatattataggtatgccatattatttttgtggcgagataacagatagacgagacaggagagctcttgccgatgccggccggacccagccaatctgcgtgtgcgccgactgtgcggacacctgaatgaacgtgccggagtacctgacccggaccgcagtcgcttcagctcaactgatggagtgtgcagtggcaacggaaaagctccatttaacgggggtcggggcgtaatgctgcgtacggcggcacggtggtagtgatacacaccgaagcacccaggaaagcatcctggctcctgtccctcctcgccatttcacgagctcccatttcacacgctcactccgtcactcgctcgcctccgccgtcgcaaagcctgccggaaccggcttgtgcggtcagttccggtgccgcgcagggcctgaggaatgccgcactccgtcctggcccgtctgccccccggttccgtccggctcgtagctgccttcgggctgctgctcctcgtctcgctgctcgttctccaccgccgccccgggaggccccatgtggcggcggccgccgccagcgaccgactcactgatccctcccggtcccggctgttcctgtcgcagtcccccggggccaacgcgagcatcgcggctgacctccgcgcgctcaccgcggggccgcacctcgcggggacgcctgcgtcggcgggtgcagccgcgcacgtgctcgcccgcctccgcgccgcgggactccaaaccctaacgcgcgagtacgagccgctgctctcgtaccctgggcacgcctccctcgcgctgctccggcccgacgggtccctgctcgcgcgcctgtcgctcgaggagcccgcggacgaggggcgccgcgtcgtgcctccgtaccacgcgtacgcgccgtccggcggggccgtcgcggaggcggtgttcgtcaacctcggccgcgaggaggactacgtcgtgctcgagaggcttggagtgggcgtgcgcggccgcgtcgcggtggcgcgccgcgggggtgggtaccgcggcggggttgtggcgcgcgctgcggacaagggagccgtcgccgtgctcatcgcgggcaacgcggacggcggcgtcgagagaggagtcgttcttctcggcggccccggcgatccgctcacccccgggtgggccgccaccagcggtgcggagcgtttgaagttcgacgataaggcagtcaagcagcggtttccgagtatcccctccatgccggtgtcggccaagacggcagcagcgatcattcgaagcctgggcggtccggccataccggcggaatggaaggacggcctcggggtggataccggtggtcttggacctggccccacactggtcaacttcacgtatcaggtataacttgctgtcttgtttctgtatgatggagtgacaacaacacgacatgtataaggacaaatttaatggacagctagatccaaaagactatgcggatttgctatgttgttgtggagtcatatggcatgtttttggtgtctcgattgggaactggatgtaggcagtgagttgacgttagcttaatatggtattgcttgcttcctctgttatatctttctggattgtcagtctatatttgtgtgcttccatctgtacaatatccatggatgtctgatacctaaattattccatctaaatttttccataggctacaattaggtgagaaatttgtaatttgattggtgaagttcattaactgattttcttcaacttaagacattccagcttaagaagggtaggcctggtgaggtggtgagagctgtctcactgagtcattaggtcacgttttcgaaacagcctctccgcatttgcggggaaggcttgcacctcattcatgtaagagccttcgacactggatctaccttaataaattttgtttgtgtggaacgtaatatggtgtttgatatgagaaaccatcctatcctagatgaggtggtgcatcatgattttatctcacgaattttggtggaatcagctcattcttcatgactatactagtattagctcatgggggatgaggtggtgatggatcaaccaattctattccatgaaccaaacaaaaaaaaagtgaggagtgagaagatggaccacccattcctcaaaccaaatatccctaagttctgcataccatatgtagtaagtggtgagcctggcttagtataattaggaaacaccataataattaaactatttacagttgaggcagcaatgaaaggtcaataaagccgtaaaggaatgttggtgttcgcttgttgtcatggcagtagcgtgttagattgtgtgtgatgttgggacttgggagggtgggccatgtttggtctaggaaatgggtgggttacagagttatcagatcctaatacctgaatcaattttcttgtttagtttgtaaaatgcatgaattgttcaaccagcatgccaatctacatgactatgatgccagtagaagatgaggaatagtgtgtgctattaaatggatcatatctaccagcagtggaccaaccgcttactaggatggagccctttcctgaccaaatctagagctatgaacaaacatttaaatgccattgttttgcatgcagtatgttttctggaatcttttgttgttgacatttatgcatttgtgttgcgcttattctgtactatgcccttgtcatcatgtacttgaaccaggtgaccttttaaatgcatgtctgtccataaatccatatcttataaggtttttgcttttgtgaagtataatttgttcataccaacagtatcttagaatgcaagtttccattattgttacctgtggtatggaatgtacatgattatgtgctttagacttccatattgctgcatgttctgtgtaaatacttttgtacatgtactttatagatgaatacactatgattgctattatctctttgtttaaggaggacaggaagttttataagatacgagacatttttggtatcataaaagggcaagaggaacctgaccgatatgtgatacttggtaaccacagagatgcatggacctatggagcagttgatcctaacagcgggacagctgcacttctagacattgctcggcgtcttggaataatgctgcaatcaggatggaaaccacggaggtccatcattctttgtagttgggacggtgaagaatttgggatggtaagtagacatggaagaaagatgataagatgctactgtataaacatgacgtataactgcaaatggaccaagagatgcttttaacaatttgtcattttattgtttaaatcacaaacgacacaactaccttaatatttgggatatgttgtcttttgactcatgacaatacacaggctatgctaaatagcagtgttgcagttaaattgttcttgcaaatcaatatatcagaataaggtggtttttggatattttcctgcatgtagttcccctatactgtggaatgcccccttcttttttcttttgctgttagattaggtcaactttgtatgtgctatctaattgtcagcacatatacttgttacttttattgtatataatatgaaggctctttttcgtccccagaattggatcaactgaatgggttgaagacaaccttgaagatctgcattccaaagctgtagcttacttgaatgttgattgtgctgtgcaaggtgtgggattttttgctggttctactccccagttggacaaactcttggttgatattacaagacaggtagaagcattttttaattgcactgttgcatttttctggttttggtctagacaagtagaaactagctaagttttgaagtcttgttgaagcaaaactatttattataggacaaatttaacagcatcatcatcatcaaaccttttgcatatgtctggaacaacttaacttgaggcattgatcaatagtagtactcaattataatgttaataccattgtctgctgtatggctaagccagattgccagaatgaatacatgatctgggattatatttaaaactattgtttcccaggtaaaaggctttgttgttgtttgtttcccaggtaagggatcctgatgtcacgggaaagatggttcatgatacatggaatgaaatgagtggcggcatcaaagtaagaattaaattttttttgaaatttcgttctgcatatgtagagattgttctcacaattatctagcattgtcatgtagttctagttgcaaaatggttattcagttacaccacagctgagccacatcgagcataatttgttaattgtagtttctgtattgctttgtcgccccccgcaggtcgagtttttttgtactgctttgtcgaacaggaacattttgtattgtgatggtagtagaggcaggaatgtactttcttacttgagttgcctgcctcatgtaaatcccacttggaggagggcatatgctcccatcattcctcttgatcttgctatgtcttgccccccactaccaccaagaaagaagaaagaacatgcttggtttctgcatgtgactaccctattcttttgctttatgataatcttgtccttatttcatgactggctttagagtttagaccaagactctgtttggcatggctccaactctaaactccatattgtagttgagtagagccatccaacaccccaactccacatttctttggagcagcctaactccatggagttggtagagctctagttcattttgagtctggagtttggagcacctcttttacaactccaaaccacttcatgatagacgtagacatggagttcgattgtatttacctttacccaccaataccatcgattacacaaaaaataattcgttttatttcctgccaaccctttctttcttccgtacactcgtgtctcttcgttgcggtatccgctagagctcctaccgtgcgcgccccgccctagttgttgcccgtcgacggtccccttcccccaccgtcagcccctgccaaagcgcccacgtcttcgagggcacacacgcctcgctcccagccgcgactatagcccgccaaggtcctcggggcctcgctctctttttgcatcggtagatcaagactcaagatcatgtacccttccttcctcttcctgtttctcatgtacgagccctgttgttttgttgcgcatacagactagatccaggaatgctctacctttacgtgatcaaaatccatggagtttgttgcgtttaatttagtgtgaagtcttgaaattacttattagacctatataactatttcactcaaaatggaaggaaggacatgaattttaaagcagacacaaagaggtataattgtttcactcaaaatggaacgaagggcatgaattttaaagcagacgcaaaaacctctttcttagagttagggacaccctgctataggtctacatttgggccgcccgacccggtccggctcaaacccgaaaaagcccgacacgtttaaatttcgggccgacacgacccataattgttacgggccgtgccgggctagcctgtgggcttagtcctcggcccacggcccgtcacgtaattgcttaaacgtgtcgggtttattttgggcggcccgaatttataaaaccccgaaattcaatttctgccctgaaattcacatcggagcccaaaattaaaacaagatttaataaaaaaataataaaagttaagataaacaaatttgaccaaaagaaaacttaatgtttctattaagttactagagtagagatgtgcaatgactgcctcgttaaaaaacttttgttagaagggaaaacaagtacaaccaactctgatgttcataacaaaagtaaaattacatcacatcctctaattcaaagctgcaaaagaaaatctaactagcataaacttcagcatcgcgttctttaagtacatggaagtgcggaatgaagtgtggttttatttaataaatatatgagccctttcgtgtctctatatggtccatttcatgcctgccttaaacgggttgtgctcgcgcctgtgggccgtgacctcggctcaagaccggcccgatgcatcgggccgggctaggtcgacactaaaatattttgggccgggtcgtgcttgggccgtgcttttttcgtgtttcaggccagcccatcagactcaacccaaatgtacagctatacaccctgctagccaaacacctacttatagatagctccaactccacgtggagcctgctccacctagagttgtagagtggagtagaaacatgtatagttggagccatgccaaaaaggggttagttttcatagttgtttcaaccaagtaacttacagcttttccacaacacttctacagctaagagctcttcacaaaaaggactcgatgattttgagtcactgatcactgcttgccttaaatattttttaggggaaaatgcgaacacccgggccaagtcaagacattgcttgtcttatatttgtttgccctatttctaaagaatgtcaaatctgcagatagaaaggcttgccagaactgattccgattttgctccatttctacatcatgctggaattccatctgtagacttgtactacggagaaggtatgtcaaaacagaataggttctttccttaatgtcccgtttggatcgttggaattgaattccattctaataataataattttggcatatatcaattaagctaatttgattttatacaaaatatatttgtatactattattagcaagatatcggagatatttatgtgctacatttttactatagaggagtgagacgaagagtgtcatgtaagttacagagtagaaacaaattctactaatgcataaaatcatttctcatcctccacctccatgaatttgagataggcatatatctgaactttggaaagtggtgggatgtcaaattccaaactaaataagttactttattgagtgaatttcaattcctctaaaatgaagggatccaaacggcccgtaagggaaaaataacatattggagatcagacatgttcaaaaggtagttgctaatgtatatttattctgctggagcttcaatttaggatcattgcaactcaaccattttgaagtcttttgtctgagctgtacatacaattttgaggctatgtacacctatagttatttcttaatatttcctttatcgcgtcagtttcaagttcgcacctacaatttctcctttaactctaatttgttctatcacgtgtcatacaagttattctaacatggtgtatggtcatcattactaaccaatgcagtgctaatgcagattttcctggttaccatactgctcttgacacttataattggatggaaaagcatggcgatccattctttcttcgtcatttggctagtaagtaaaaacataatatatgcactgatacaaaaagggtcaacaagaagaatgaactgttgcctgtaaagatagctactaaatactcaacaatttttgaaattttagtgtgccaaaataatgtattgctggcatagcatttgttgtggtaatcctaggagtgcttatatagtttttctgcttcatcagtcacagaaatttggggactattagctcttcgattggccaatgatcctgtgttaccttttgattatcaagcttacacttcgcagttacaggtacattgactttcatatgcatactagatttctacattgcaaaggtgtattgcttggagaataccacctagtagtgtgacatgagtgacattggttgaatctgtaacttcagtgttttttttttgtaatatggagatgtacatttgagtaaatcttatgtacgagaactcacacattgcataataccaccaatttgctttcagtcattattatggcgtatcttacgatatatatttaatatgattgaaatttgtaatgccacaggagcatataaaaacattgtctgccctgacgagcgatggccatgcagtaaatttgatgaatgggtgcgtcaatgacctttctggtgcagctatggaagttctgaaggagatgaaggtatcaatttatgccgtgcttctgatttcatggattttagggtcggtctattttcacaatctggttgtcaatggaatattctttttttcgatttataagtggtgtttgaaactggctgatgacgtgctccgttgtatgttcggattcaaaatacagaaactacagcagatggatttatatgatgaacacaccaggatgagaaggcgattgttgaacgatcgcctcctacttgctgaaagaagcttcctgcaacctgaagggctccaaggaagaggatggtttaagcatctggtgagaaacgcatttcagcggacttgacgacttgtatattattttcctttaaactaaaacttgtttttcaccagttgtactcgcctccggaggactacgaaagcaagctgtcgttcttccctgggatcgctgacgccatctcacggtccgccaatctgagtgacaaggaacaggaggtggcaatgcagcatgaagtgtggaaagtctgccgtgcgattcaaagggctgcgagtgttcttagaggtgaattcagtgagcaaaaaccgacgaattttagttctttggtgaccccatgagatgtgtttgtgcaaacaaataacaaacaatggttatttagtatctgcgcgaaatggaagtgtgatgaagaatgtga

SEQ ID NO: 18 - DNA, 27 bp, Artificial Sequence
  Oligonucleotide primer 171muF
tgttcgtcaacctcggccgcgaggagg

SEQ ID NO: 19 - DNA, 27 bp, Artificial Sequence
  Oligonucleotide primer 63289
aaaccgctgcttgactgccttatcgtc

SEQ ID NO: 20 - DNA, 32 bp, Artificial Sequence
  Oligonucleotide primer 9242 mu tir
agagaagccaacgccawcgcctcyatttcgtc

SEQ ID NO: 21 - DNA, 23 bp, Artificial Sequence
  Oligonucleotide primer 63288
gaaccggcttgtgcggtcagttc

SEQ ID NO: 22 - DNA, 6157 bp, Artificial Sequence
  pML18
gaatatgcatcactagtaagctttgctctagactggaattcgtcgactctagaggatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaatagtgagatccaacacttacgtttgcaacgtccaagagcaaatagaccacgnacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggtactgtgtaatgacgatgagcaatcgagaggctgactaacaaaaggtacatcggtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgcagatttaggtgacactata

(19) United States
(12) Patent Application Publication
(10) Pub. No.: US 2010/0199369 A1
(43) Pub. Date: Aug. 5, 2010

(54) Alteration of Plant Embryo/Endosperm Size During Seed Development

(76) Inventors: Sakai Hajime, Newark, DE (US); Graziana Taramino, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/083,820

(22) PCT Filed: Dec. 15, 2006
(86) PCT No.: PCT/US06/47998

§ 371 Date: Apr. 16, 2008

Related U.S. Application Data
(60) Provisional application No. 60/751,028, filed on Dec. 16, 2005.

(51) Int. Cl. A01H 5/00; C07H 21/04; C12N 15/63; C12N 15/82; A01H 1/00

(52) U.S. Cl. 800/260; 536/23.6; 435/320.1; 800/298; 800/312; 800/320.2; 800/320.1; 800/320.3; 800/320; 435/468; 800/287

(57) ABSTRACT

Isolated polynucleotides and recombinant constructs comprising such fragments useful for altering embryo/endosperm size during seed development are disclosed along with a method of controlling embryo/endosperm size during seed development in plants using such recombinant constructs.

### FIELD OF THE INVENTION

The present invention is in the field of plant breeding and genetics and, in particular, relates to polynucleotides, that when mutated, alter embryo/endosperm size during seed development, recombinant constructs useful for altering embryo/endosperm size during seed development as well as a method of controlling embryo/endosperm size during seed development in plants using such recombinant constructs.

### BACKGROUND OF THE INVENTION

Elucidation of how the size of a developing embryo is genetically regulated is important because the final volume of endosperm as a storage organ of starch and proteins is affected by embryo size in cereal crops. Researchers have found that genes involved in embryo size contribute to the regulation of endosperm development. Investigation of these genes is important for agriculture because cereal endosperms are the staple diet in many countries.

The giant embryo (ge) mutation was first described by Satoh and Omura (1981) Jap. J. Breed. 31:316-326. The giant embryo mutant is a potentially useful character for quality improvement in cereals because increased embryo size will result in increased embryo oil and nutrient traits that are desirable for human consumption. Also, the enlargement of embryos would result in increased embryo-related enzymatic activities, which are often important features in the processing of grains. The mutation was genetically mapped to chromosome 7 (Iwata and Omura (1984) Japan. J. Genet. 59: 199-204; Satoh and Iwata (1990) Japan. J. Breed. 40 (Suppl. 2): 268-269), with additional ge alleles also localized to chromosome 7 (Koh et al. (1996) Theor. Appl. Genet. 93:257-261). The ge mutations were analyzed at the morphologic and genetic level by Hong et al. (1994) Development 122:2051-2058. This publication linked the GE gene as being required for proper endosperm development.

Since both endosperm and embryo size are affected by the mutation, GE appears to control coordinated proliferation of the endosperm and embryo during development. Beside the morphological change of embryo and endosperm in ge, it was also shown that the ge seed accumulates more oil compared to the wild type (Matsuo et al. (1987) Japan. J. Breed. 37: 185-191; Okuno (1997) In “Science of the Rice Plant” Vol. III, Matsuo et al. eds., Food and agriculture policy research center, Tokyo, Japan, pp 433-435).

It was found that loss-of-function of the GE gene leads to an enlargement of embryonic tissue at the expense of endosperm tissue. This developmental change may be useful in increasing the amount of embryo-specific metabolites such as oil in seed-bearing plants. The GE gene constitutes the subject matter of Applicants' Assignee's PCT Publication WO 02/099063 published Dec. 12, 2002.

The present invention expands the understanding of genetic regulation of embryo/endosperm size during seed development. Specifically, a new single gene recessive mutant has been identified and named goliath (go).

### SUMMARY OF THE INVENTION

In a first embodiment, the invention relates to an isolated polynucleotide comprising:
(a) a nucleic acid sequence encoding a polypeptide involved in altering embryo/endosperm size during seed development, said polypeptide having at least 80% amino acid sequence identity, based on the Clustal V method of alignment, when compared to an amino acid sequence selected from the group consisting of SEQ ID NOs:12 and 16; or
(b) the nucleic acid sequence set forth in SEQ ID NO:11 wherein said sequence comprises at least one of the following modifications:
   (i) nucleotide 5103 is a T residue instead of a C; or
   (ii) nucleotides 4511 through 4540 have been deleted; or
(c) the nucleic acid sequence set forth in SEQ ID NO:11, 13; 15, or 17; or
(d) all or part of the isolated polynucleotide comprising sequences of (a), (b), or (c) for use in suppression of endogenous nucleic acid sequences encoding polypeptides involved in altering embryo/endosperm size during seed development; or
(e) the full complement of (a), (b), (c); or (d).

In a second embodiment, the invention relates to an isolated polynucleotide encoding a encoding a polypeptide involved in altering embryo/endosperm size during seed development wherein said Isolated polynucleotide hybridizes under stringent conditions to one of the nucleotide sequences set forth in SEQ ID NOs:11, 13; 15, and 17.

In a third embodiment, the invention relates to an isolated polynucleotide comprising a nucleotide sequence encoding a polypeptide involved in altering embryo/endosperm size during seed development, wherein the nucleotide sequence has at least 80% sequence identity, based on the BLASTN method of alignment, when compared to a nucleotide sequence as set forth in SEQ ID NOs:11, 13; 15, and 17.

In a fourth embodiment, the invention relates to recombinant DNA construct comprising the isolated polynucleotide of the invention operably linked to at least one regulatory sequence.

In a fifth embodiment, the invention relates to a plant comprising in its genome the recombinant DNA construct of the invention as well as any seeds obtained from such plant and the oil obtained from such seeds. Also of interest are transformed plant cells or plant tissue comprising the recombinant DNA construct of the invention.

In a sixth embodiment the invention relates to a method of altering embryo/endosperm size during seed development in a plant comprising:
(a) transforming plant cells or plant tissue with the recombinant DNA construct of the invention;
(b) regenerating transgenic plants from the transformed plant cells or plant tissue of (a);
(c) screening the transgenic plants of (b) for seeds having an altered embryo/endosperm size based on a comparison of embryo/endosperm size of seeds obtained from non-transformed plants.

In a seventh embodiment, the invention relates to a method of mapping genetic variations related to controlling embryo/endosperm size and/or altering oil phenotype in plants comprising:
(a) crossing two plant varieties; and (b) evaluating genetic variations with respect to
   (i) a nucleic acid sequence selected from the group consisting of SEQ ID NOs:11, 13, 15, and 17; or
   (ii) a nucleic acid sequence encoding a polypeptide selected from the group consisting of SEQ ID NOs:12 and 16; in progeny plants resulting from the cross of step (a) wherein the evaluation is made using a method selected from the group consisting of RFLP analysis, SNP analysis, and PCR-based analysis.

In an eighth embodiment, the invention relates to a method of molecular breeding to control embryo/endosperm size and/or altering oil phenotype in plants comprising:
(a) crossing two plant varieties; and
(b) evaluating genetic variations with respect to
   (i) a nucleic acid sequence selected from the group consisting of SEQ ID NOs:11, 13, 15, and 17; or
   (ii) a nucleic acid sequence encoding a polypeptide selected from the group consisting of SEQ ID NOs:12 and 16; in progeny plants resulting from the cross of step (a) wherein the evaluation is made using a method selected from the group consisting of RFLP analysis, SNP analysis, and PCR-based analysis.

### BRIEF DESCRIPTION OF THE SEQUENCE LISTING

The invention can be more fully understood from the following detailed description and the accompanying Sequence Listing which form a part of this application.

The sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence of oligonucleotide primer SSR 45F.

SEQ ID NO:2 is the nucleotide sequence of oligonucleotide primer SSR 45R.

SEQ ID NO:3 is the nucleotide sequence of oligonucleotide primer C3-145F.

SEQ ID NO:4 is the nucleotide sequence of oligonucleotide primer C3-145R.

SEQ ID NO:5 is the nucleotide sequence of oligonucleotide primer b0060j21ssr1.

SEQ ID NO:6 is the nucleotide sequence of oligonucleotide primer b0060j21 ssr2.

SEQ ID NO:7 is the nucleotide sequence of oligonucleotide primer b0024J04ssr3.

SEQ ID NO:8 is the nucleotide sequence of oligonucleotide primer b0024J04ssr4.

SEQ ID NO:9 is the nucleotide sequence of oligonucleotide primer A87o09ssr1.

SEQ ID NO:10 is the nucleotide sequence of oligonucleotide primer A87o09ssr2.

SEQ ID NO:11 is the genomic nucleotide sequence of the rice GO gene.

SEQ ID NO:12 is the amino acid sequence deduced from translating nucleotides 4152-5102, 6002-6244, 6530-6682, 6828-3896, 9134-9221, 9600-9683, 9947-10035, 10386-10499, 10966-11100, and 11300-11524 from SEQ ID NO:11.

SEQ ID NO:13 is the nucleotide sequence of the cDNA insert from clone rls6.pk0079.c3 encoding a rice GO gene.

SEQ ID NO:14 is the amino acid sequence obtained from translating nucleotides 100 through 2250 of SEQ ID NO:13.

SEQ ID NO:15 is the nucleotide sequence of the corn ortholog of GO obtained from cDNA insert from clone csc1c.pk003.k10 (nucleotides 568-2260) linked to the remaining portion of exon 1 obtained from the BAC clone BACM2.pk146.m06 (nucleotides 1-567).

SEQ ID NO:16 is the amino acid sequence derived from nucleotides 1 through 2139 of SEQ ID NO:15.

SEQ ID NO:17 is the nucleotide sequence of the genomic insert in clone BACM2.pk146.m06.

SEQ ID NO:18 is the nucleotide sequence of oligonucleotide primer 171muF.

SEQ ID NO:19 is the nucleotide sequence of oligonucleotide primer 63289.

SEQ ID NO:20 is the nucleotide sequence of oligonucleotide primer 9242 mu tir.

SEQ ID NO:21 is the nucleotide sequence of oligonucleotide primer 63288.

SEQ ID NO:22 is the nucleotide sequence of vector pML18.

SEQ ID NO:23 is the nucleotide sequence of oligonucleotide primer GO-xhoF1.

SEQ ID NO:24 is the nucleotide sequence of the T7-specific oligonucleotide primer used to amplify the Oryza sativa GO open reading frame.

SEQ ID NO:25 is the nucleotide sequence of binary vector OsGOBE861.

SEQ ID NO:26 is the nucleotide sequence of oligonucleotide primer GO 1566F.

SEQ ID NO:27 is the nucleotide sequence of oligonucleotide primer GO 1747R.

SEQ ID NO:28 is the nucleotide sequence of oligonucleotide primer Amp1-1566F.

SEQ ID NO:29 is the nucleotide sequence of oligonucleotide primer Amp1-1747R.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

Disclosure of all references, patents, and patent applications cited herein are hereby incorporated by reference.

The terms “isolated nucleic acid fragment” and “isolated polynucleotide” are used interchangeably herein. These terms refer to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for Inosine, and “N” for any nucleotide.

The terms “Oryza sativa GO” and “rice GO” are used interchangeably herein. These terms refer to an isolated polynucleotide isolated from wild-type rice and whose sequence is set forth herein.

The terms “Oryza sativa go” and “rice go” are used interchangeably herein. The terms refer to a mutant goliath, or go, isolated polynucleotide.

Down-regulation of the Goliath (GO) function in a homozygous plant results in enlargement of embryonic tissue. However, the size of the endosperm may be reduced or the size of the endosperm may not be altered. On the other hand, overexpression of this gene might lead to a reduction of embryonic tissue, thus, resulting in a smaller embryo size. In this case, the size of the endosperm might increase or it might not be altered.

The term “down-regulation” refers to a partial or complete suppression or silencing of a gene using techniques including, but not limited to, co-suppression, RNA interference, and anti-sense. Such techniques are discussed in greater detail below.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of recombinant DNA constructs to produce the desired phenotype in a transformed plant Recombinant DNA constructs can be designed for use in co-suppression or antisense by linking a nucleic acid fragment or subfragment thereof, whether or not it encodes an active enzyme, in the appropriate orientation relative to a plant promoter sequence.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

A “homolog” can be a second gene in the same plant type or in a different plant type that has a polynucleotide sequence that is functionally identical to a sequence in the first gene. It is believed that, in general, homologs share a common evolutionary past.

“Orthologs” are genes from different species that derive from a common ancestor and, generally, share the same function. Hence, comparative genomics frequently provides an insight into the putative functions of genes in different species, i.e., orthologs.

One skilled in the art will understand that substantially similar nucleic acid sequences encompassed by this invention are also defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, UK).

Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

“Sequence identity” or “identity” in the context of nucleic acid or polypeptide sequences refers to the nucleic acid bases or amino acid residues in the two sequences that are the same when aligned for maximum correspondence over a specified comparison window.

Thus, “Percent sequence identity” refers to the values determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any integer percentage from 55% to 100%. These identities can be determined using any of the programs described herein.

Sequence alignments and percent identity or similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal V method of alignment (Higgins, D. G. and Sharp, P. M. (1989) Comput. Appl. Biosci. 5:151-153; Higgins, D. G. et al. (1992) Comput. Appl. Biosci. 8:189-191) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

The “Clustal V method of alignment” corresponds to the alignment method labeled Clustal V (described by Higgins and Sharp, CABIOS. 5:151-153 (1989)) and found in the MegAlign™ program of the LASERGENE bioinformatics computing suite (DNASTAR Inc., Madison, Wis.). The “default parameters” are the parameters preset by the manufacturer of the program. For multiple alignments, they correspond to GAP PENALTY=10 and GAP LENGTH PENALTY=10; and, for pairwise alignments, they are KTUPLE 1, GAP PENALTY=3, WINDOW=5 and DIAGONALS SAVED=5. After alignment of the sequences using the Clustal V program, it is possible to obtain a “percent identity” by viewing the “sequence distances” table in the same program.

“BLASTN method of alignment” is an algorithm provided by the National Center for Biotechnology Information (NCBI) to compare nucleotide sequences using default parameters.

It is well understood by one skilled in the art that many levels of sequence identity are useful in identifying polypeptides, from other plant species, wherein such polypeptides have the same or similar function or activity. Useful examples of percent identities include, but are not limited to, 50%, 55%, 60%, 65%, 70%, 75%, 80%, 85%, 90%, or 95%, or any integer percentage from 55% to 100%. Indeed, any integer amino acid identity from 50%-100% may be useful in describing the present invention. Also, of interest is any full or partial complement of this isolated nucleotide fragment.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. A “foreign” gene refers to a gene not normally found in the host organism, but that is introduced into the host organism by gene transfer. Foreign genes can comprise native genes inserted into a non-native organism, or recombinant DNA constructs. A “transgene” is a gene that has been introduced into the genome by a transformation procedure.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoter sequences can also be located within the transcribed portions of genes, and/or downstream of the transcribed sequences. Promoters may be derived in their entirety from a native gene, or be composed of different elements derived from different promoters found in nature, or even comprise synthetic DNA segments. It is understood by those skilled in the art that different promoters may direct the expression of an isolated nucleic acid fragment in different tissues or cell types, or at different stages of development, or in response to different environmental conditions. Promoters which cause an isolated nucleic acid fragment to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of some variation may have identical promoter activity.

Commonly used promoters that may be useful in expressing the nucleic acid fragments of the invention include, but are not limited to, the oleosin promoter (PCT Publication WO99/65479, published Dec. 12, 1999), the maize 27 kD zein promoter (Ueda et al (1994) Mol. Cell. Biol. 14:4350-4359), the ubiquitin promoter (Christensen et al (1992) Plant Mol. Biol. 18:675-680), the SAM synthetase promoter (PCT Publication WO00/37662, published Jun. 29, 2000), the CaMV 35S promoter (Odell et al (1985) Nature 313:810-812), and the promoter described in PCT Publication WO02/099063 published Dec. 12, 2002.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form, for example, using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “endogenous RNA” refers to any RNA which is encoded by any nucleic acid sequence present in the genome of the host prior to transformation with the recombinant construct of the present invention, whether naturally-occurring or non-naturally occurring, i.e., introduced by recombinant means, mutagenesis, etc.

The term “non-naturally occurring” means artificial, not consistent with what is normally found in nature.

The term “operably linked” refers to an association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

As stated herein, “suppression” refers to the reduction of the level of enzyme or enzyme activity detectable in a transgenic plant when compared to the level of enzyme or enzyme activity detectable in a plant not transformed with a recombinant DNA of the invention. This reduction may be due to the decrease in translation of the native mRNA into an active enzyme. It may also be due to the transcription of the native DNA into decreased amounts of mRNA and/or to rapid degradation of the native mRNA. Screening to obtain lines displaying the desired phenotype may be accomplished by Southern analysis of DNA, Northern analysis of mRNA expression, RT-PCR, immunoblotting analysis of protein expression, or phenotypic analysis, among others.

“Antisense inhibition” refers to the production of antisense RNA transcripts capable of suppressing the expression of the target protein. “Antisense RNA” refers to an RNA transcript that is complementary to all or part of a target primary transcript or mRNA and that blocks the expression of a target isolated nucleic acid fragment (U.S. Pat. No. 6,107,065). The complementarity of an antisense RNA may be with any part of the specific gene transcript, i.e., at the 5′ non-coding sequence, 3′ non-coding sequence, introns, or the coding sequence. It is not necessary for the antisense RNA transcript to be 100% complementary to the target primary transcript for there to be suppression.

“Co-suppression” refers to the production of sense RNA transcripts capable of suppressing the expression of identical or substantially similar native genes (U.S. Pat. No. 5,231,020). Co-suppression constructs in plants have been previously designed by focusing on overexpression of a nucleic acid sequence having homology to a native mRNA, in the sense orientation, which results in the reduction of all RNA having homology to the sequence. Co-suppression technology constitutes the subject matter of U.S. Pat. No. 5,231,020 (for reviews see. Vaucheret et al. (1998) Plant J. 16:651-659; and Gura (2000) Nature 404:804-808). Plant viral sequences may be used to direct the suppression of proximal mRNA encoding sequences (PCT Publication WO 98/36083 published on Aug. 20, 1998).

Chimeric genes encoding sense and antisense RNA molecules comprising nucleotide sequences respectively homologous and complementary to at least a part of the nucleotide sequence of the gene of interest and wherein the sense and antisense RNA are capable of forming a double stranded RNA molecule or “Hairpin” structure have been described as capable of suppressing a gene (PCT Publication WO 99/53050 published on Oct. 21, 1999). For review of hairpin suppression see Wesley, S. V. et al. (2003) Methods in Molecular Biology, Plant Functional Genomics: Methods and Protocols 236:273-286. The use of poly-T and poly-A sequences to generate the stem in the stern-loop structure has also been described (WO 02/00894 published Jan. 3, 2002). Yet another variation includes using synthetic repeats to promote formation of a stem in the stern-loop structure (PCT Publication WO 02/00904, published Jan. 3, 2002).

The use of constructs having convergent promoters directing transcription of gene-specific sense and antisense RNAs inducing gene suppression has also been described (see for example Shi, H. et al. (2000) RNA 6:1069-1076; Bastin, P. et al. (2000) J. Cell Sci. 113:3321-3328; Giordano, E. et al. (2002) Genetics 160:637-648; LaCount, D. J. and Donelson, J. E. US patent Application No. 20020182223, published Dec. 5, 2002; Tran, N. et al. (2003) BMC Biotechnol. 3:21; and Applicant's U.S. Provisional Application No. 60/578,404, filed Jun. 9, 2004).

RNA interference (RNAi) is defined as the ability of double-stranded RNA (dsRNA) to suppress the expression of a gene corresponding to its own sequence and is the subject of U.S. Pat. No. 6,506,559, issued Jan. 14, 2003.

Other methods for suppressing an enzyme include, but are not limited to, use of polynucleotides that may form a catalytic RNA or may have ribozyme activity (U.S. Pat. No. 4,987,071 issued Jan. 22, 1991).

“Overexpression” refers to the production of a functional end-product in transgenic organisms that exceeds levels of production when compared to expression of that functional end-product in a normal, wild type, or non-transformed organism, or an organism not-transformed with a recombinant DNA fragment comprising a polynucleotide of the invention.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The preferred method of cell transformation of rice, corn and other monocots is using particle-accelerated or “gene gun” transformation technology (Klein et al. (1987) Nature (London) 327:70-73; U.S. Pat. No. 4,945,050), or an Agrobacterium-mediated method (Ishida Y. et al. (1996) Nature Biotech. 14:745-750). The term “transformation” as used herein refers to both stable transformation and transient transformation.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press Cold Spring Harbor, 1989.

The term “recombinant” refers to an artificial combination of two otherwise separated segments of sequence, e.g., by chemical synthesis or by the manipulation of isolated segments of nucleic acids by genetic engineering techniques.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

Polymerase chain reaction (“PCR”) is a powerful technique used to amplify DNA millions of fold, by repeated replication of a template, in a short period of time. (Mullis et al. (1986) Cold Spring Harbor Symp. Quant. Biol. 51:263-273; Erlich et al, European Patent Application 50,424; European Patent Application 84,796; European Patent Application 258,017, European Patent Application 237,362; Mullis, European Patent Application 201,184, Mullis et al U.S. Pat. No. 4,683,202; Erlich, U.S. Pat. No. 4,582,788; and Saiki et al, U.S. Pat. No. 4,683,194). The process utilizes sets of specific in vitro synthesized oligonucleotides to prime DNA synthesis. The design of the primers is dependent upon the sequences of DNA that are to be analyzed. The technique is carried out through many cycles (usually 20-50) of melting the template at high temperature, allowing the primers to anneal to complementary sequences within the template and then replicating the template with DNA polymerase.

The products of PCR reactions are analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

The terms “recombinant construct”, “expression construct” and “recombinant expression construct” are used interchangeably herein. These terms refer to a functional unit of genetic material that can be inserted into the genome of a cell using standard methodology well known to one skilled in the art. Such construct may be itself or may be used in conjunction with a vector. If a vector is used then the choice of vector is dependent upon the method that will be used to transform host plants as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA; Northern analysis of mRNA expression, Western analysis of protein expression, or phenotypic analysis.

The instant invention concerns, in one embodiment, an isolated polynucleotide comprising:
(a) a nucleic acid sequence encoding a polypeptide involved in altering embryo/endosperm size during seed development, said polypeptide having at least 80% amino acid sequence identity based on the Clustal V method of alignment, when compared to an amino acid sequence selected from the group consisting of SEQ ID NOs:12 and 16; or
(b) the nucleic add sequence set forth in SEQ ID NO:11 wherein said sequence comprises at least one of the following modifications; or
   (i) nucleotide 5103 is a T residue instead of a C; or
   (ii) nucleotides 4511 through 4540 have been deleted;
(c) the nucleic acid sequence set forth in SEQ ID NO:11, 13; 15, or 17; or
(d) all or part of the isolated polynucleotide comprising sequences of (a), (b); (c) or (d) for use in suppression of endogenous nucleic acid sequences encoding polypeptides involved in altering embryo/endosperm size during seed development; or
(e) the full complement of (a), (b), (c); or (d).

The rice GO gene of the present invention was identified through map-based cloning and the corn GO ortholog of the present invention was identified by sequence comparison and evaluation of its activity. These GO genes were found to share sequence identity with an Arabidopsis carboxypeptidase gene which in turn shares sequence identity with an Arabidopsis thaliana AMP1 gene. Given this sequence similarity, it appears that the GO gene encodes a carboxypeptidase.

It was also found that the GO mutants are recessive. Thus, one copy of the mutant GO gene in a heterozygous plant has no effect on embryo size. However, down-regulation of both copies of the GO gene in a homozygous plant produces seeds having an enlarged embryo.

Accordingly, the enlarged embryo phenotype is associated with a change in the wild type GO sequence that results in loss of function of the GO gene and the concomitant change in embryo size. Support for this is set forth in Examples 1 and 2 below.

The term “homozygous” in a diploid organism refers to an organism that carries two identical copies of the same allele. A “recessive” allele is one that is not expressed when in the presence of the “dominant” allele, i.e., two copies of a recessive allele are needed in order for a recessive gene to be expressed.

As was noted above, the rice polynucleotide comprising the GO gene was identified in the instant application using high fidelity mapping of DNA obtained from goliath (go) mutants. These mutants have an enlarged embryo phenotype.

In a second embodiment, the invention relates to an isolated polynucleotide encoding a encoding a polypeptide involved in altering embryo/endosperm size during seed development wherein said isolated polynucleotide hybridizes under stringent conditions to one of the nucleotide sequences set forth in SEQ ID NOs:11, 13; 15, and 17.

One skilled in the art will understand that substantially similar nucleic acid sequences encompassed by this invention are also defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Estimates of such homology are provided by either DNA-DNA or DNA-RNA hybridization under conditions of stringency as is well understood by those skilled in the art (Hames and Higgins, Eds. (1985) Nucleic Acid Hybridisation IRL Press, Oxford, UK).

Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

In a third embodiment, the invention relates to an isolated polynucleotide comprising a nucleotide sequence encoding a polypeptide involved in altering embryo/endosperm size during seed development, wherein the nucleotide sequence has at least 80% sequence identity, based on the BLASTN method of alignment, when compared to a nucleotide sequence as set forth in SEQ ID NOs:11, 13; 15, and 17.

In a fourth embodiment, the invention relates to recombinant DNA construct comprising the isolated polynucleotide of the invention operably linked to at least one regulatory sequence. Those skilled in the art will appreciated that the nucleotide sequences described herein can be operably linked to at least one regulatory sequence in a sense or antisense orientation.

Such constructs can then be used to transform plants, plant tissue, or plant cells. Transformation methods are well known to those skilled in the art and are described herein. Any plant, dicot or monocot, can be transformed with recombinant DNA constructs of the invention.

Examples of monocots include, but are not limited to, corn, wheat, rice, sorghum, millet, barley, palm, lily, Alstroemeria, rye, and oat.

Examples of dicots include, but are not limited to, soybean, rape, sunflower, canola, grape, guayule, columbine, cotton, tobacco, peas, beans, flax, safflower, and alfalfa.

Preferably, the plant can be selected from the group consisting of rice, corn, sorghum, millet, rye, soybean, canola, wheat, barley, oat, beans, and nuts.

Plant tissue includes differentiated and undifferentiated tissues or plants, including but not limited to, roots, stems, shoots, leaves, pollen, seeds, tumor tissue, and various forms of cells and culture such as single cells, protoplasm, embryos, and callus tissue. The plant tissue may in plant or in organ, tissue or cell culture.

The term “plant organ” refers to plant tissue or group of tissues that constitute a morphologically and functionally distinct part of a plant. The term “genome” refers to the following: 1. The entire complement of genetic material (genes and non-coding sequences) is present in each cell of an organism, or virus or organelle. 2. A complete set of chromosomes inherited as a (haploid) unit from one parent. The term “stably integrated” refers to the transfer of a nucleic acid fragment into the genome of a host organism or cell resulting in genetically stable inheritance.

Also within the scope of this invention are seeds obtained from such transformed plants and oil obtained from such seeds.

In another aspect, this invention relates to a method of altering embryo/endosperm size during seed development in a plant comprising:
(a) transforming plant cells or plant tissue with a recombinant DNA construct of the invention;
(b) regenerating transgenic plants from the transformed plant cells or plant tissue of (a);
(c) screening the transgenic plants of (b) for seeds having an altered embryo/endosperm size based on a comparison with embryo/endosperm size of seeds obtained from non-transformed plants.

The regeneration, development, and cultivation of plants from single plant protoplast transformants or from various transformed explants is well known in the art (Weissbach and Weissbach, In: Methods for Plant Molecular Biology, (Eds.), Academic Press, inc. San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily using Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011, McCabe et. al. (1988) Bio/Technology 6:923, Christou et al. (1988) Plant Physiol. 87:671-674); Brassica (U.S. Pat. No. 5,463,174); peanut (Cheng et al. (1996) Plant Cell Rep. 15:653-657, McKently et al. (1995) Plant Cell Rep. 14:699-703); papaya and pea (Grant et al. (1995) Plant Cell Rep. 15:254-258).

Transformation of monocotyledons using electroporation, particle bombardment, and Agrobacterium have also been reported. Transformation and plant regeneration have been achieved in asparagus (Bytebier et al., Proc. Natl. Acad. Sci. (USA) (1987) 84:5354); barley (Wan and Lemaux (1994) Plant Physiol. 104:37); Zea mays (Rhodes et al. (1988) Science 240:204, Gordon-Kamm et al. (1990) Plant Cell 2:603-618, Fromm et al. (1990) Bio/Technology 8:833; Koziel et al. (1993) Bio/Technology 11: 194, Armstrong et al. (1995) Crop Science 35:550-557); oat (Somers et al. (1992) Bio/Technology 10:15 89); orchard grass (Horn et al. (1988) Plant Cell Rep. 7:469); rice (Toriyama et al. (1986) Theor. Appl. Genet. 205:34; Part et al. (1996) Plant Mol. Biol. 32:1135-1148; Abedinia et al. (1997) Aust. J. Plant Physiol. 24:133-141; Zhang and Wu (1988) Theor. Appl. Genet. 76:835; Zhang et al. (1988) Plant Cell Rep. 7:379; Battraw and Hall (1992) Plant Sci. 86:191-202; Christou et al. (1991) Bio/Technology 9:957); rye (De la Pena et al. (1987) Nature 325:274); sugarcane (Bower and Birch (1992) Plant J. 2:409); tall fescue (Wang et al. (1992) Bio/Technology 10:691), and wheat (Vasil et al. (1992) Bio/Technology 10:667; U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)).

Transient expression systems may be used to functionally dissect isolated nucleic acid fragment constructs (see generally, Maliga et al., Methods in Plant Molecular Biology, Cold Spring Harbor Press (1995)). It is understood that any of the nucleic acid molecules of the present invention can be introduced into a plant cell in a permanent or transient manner in combination with other genetic elements such as vectors, promoters, enhancers etc.

In addition to the above discussed procedures the standard resource materials which describe specific conditions and procedures for the construction, manipulation and isolation of macromolecules (e.g., DNA molecules, plasmids, etc.), generation of recombinant organisms and screening and isolating of clones (see for example, Sambrook et al., Molecular Cloning: A Laboratory Manual, Cold Spring Harbor Press (1989); Maliga et al., Methods in Plant Molecular Biology, Cold Spring Harbor Press (1995); Birren et al., Genome Analysis: Detecting Genes, 1, Cold Spring Harbor, N.Y. (1998); Birren et al., Genome Analysis: Analyzing DNA, 2, Cold Spring Harbor, N.Y. (1998); Plant Molecular Biology: A Laboratory Manual, eds. Clark, Springer, New York (1997)) are well known.

In still another aspect, this invention concerns a method of mapping genetic variations related to controlling embryo/endosperm size during seed development and/or altering oil phenotypes in plants comprising:
(a) crossing two plant varieties; and
(b) evaluating genetic variations with respect to a nucleic acid sequence selected from the group consisting of SEQ ID NOs:11, 13, 15, and 17; or a nucleic acid sequence encoding a polypeptide selected from the group consisting of SEQ ID. NOs:12 and 16; in progeny plants resulting from the cross of step (a) wherein the evaluation is made using a method selected from the group consisting of RFLP analysis, SNP analysis, and PCR-based analysis.

The terms “mapping genetic variation” or “mapping genetic variability” are used interchangeably and define the process of identifying changes in DNA sequence, whether from natural or induced causes, within a genetic region that differentiates between different plant lines, cultivars, varieties, families, or species. The genetic variability at a particular locus (gene) due to even minor base changes can alter the pattern of restriction enzyme digestion fragments that can be generated. Pathogenic alterations to the genotype can be due to deletions or insertions within the gene being analyzed or even single nucleotide substitutions that can create or delete a restriction enzyme recognition site. Restriction fragment length polymorphism (RFLP) analysis takes advantage of this and utilizes Southern blotting with a probe corresponding to the isolated nucleic acid fragment of interest.

Thus, if a polymorphism (i.e., a commonly occurring variation in a gene or segment of DNA; also, the existence of several forms of a gene (alleles) in the same species) creates or destroys a restriction endonuclease cleavage site, or if it results in the loss or insertion of DNA (e.g., a variable nucleotide tandem repeat (VNTR) polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms: (“RFLPs”). RFLPs have been widely used in human and plant genetic analyses (Glassberg, UK Patent Application 2135774; Skolnick et al, Cytogen. Cell Genet. 32:58-67 (1982); Botstein et al, Ann. J. Hum. Genet. 32:314-331 (1980); Fischer et al (PCT Application WO 90/13668; Uhlen, PCT Application WO 90/11369).

A central attribute of “single nucleotide polymorphisms” or “SNPs” is that the site of the polymorphism is at a single nucleotide. SNPs have certain reported advantages over RFLPs or VNTRs. First, SNPs are more stable than other classes of polymorphisms. Their spontaneous mutation rate is approximately 10⁻⁹ (Kornberg, DNA Replication, W.H. Freeman & Co., San Francisco, 1980), approximately, 1,000 times less frequent than VNTRs (U.S. Pat. No. 5,679,524). Second, SNPs occur at greater frequency, and with greater uniformity than RFLPs and VNTRs. As SNPs result from sequence variation, new polymorphisms can be identified by random sequencing of genomic or cDNA molecules. SNPs can also result from deletions, point mutations and insertions. Any single base alteration, whatever the cause, can be a SNP. The greater frequency of SNPs means that they can be more readily identified than the other classes of polymorphisms.

SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism or by other biochemical interpretation. SNPs can be sequenced by a number of methods. Two basic methods may be used for DNA sequencing, the chain termination method of Sanger et al, Proc. Natl. Acad. Sci. (U.S.A.) 74:5463-5467 (1977), and the chemical degradation method of Maxam and Gilbert, Proc. Natl. Acad. Sci. (U.S.A.) 74: 560-564 (1977).

Furthermore, single point mutations can be detected by modified PCR techniques such as the ligase chain reaction (“LCR”) and PCR-single strand conformational polymorphisms (“PCR-SSCP”) analysis. The PCR technique can also be used to identify the level of expression of genes in extremely small samples of material, e.g., tissues or cells from a body. The technique is termed reverse transcription-PCR (“RT-PCR”).

In another embodiment, this invention relates to a method of molecular breeding to obtain altered embryo/endosperm size during seed development and/or altered oil phenotypes in plants comprising:
(a) crossing two plant varieties; and
(b) evaluating genetic variations with respect to:
   (i) a nucleic acid sequence selected from the group consisting of SEQ ID NOs:11, 13, 15, and 17; or
   (ii) a nucleic acid sequence encoding a polypeptide selected from the group consisting of SEQ ID NOs:12 and 16; in progeny plants resulting from the cross of step (a) wherein the evaluation is made using a method selected from the group consisting of RFLP analysis, SNP analysis, and PCR-based analysis.

The term “molecular breeding” defines the process of tracking molecular markers during the breeding process. It is common for the molecular markers to be linked to phenotypic traits that are desirable. By following the segregation of the molecular marker or genetic trait, instead of scoring for a phenotype, the breeding process can be accelerated by growing fewer plants and eliminating assaying or visual inspection for phenotypic variation. The molecular markers useful in this process include, but are not limited to, any marker useful in identifying mapable genetic variations previously mentioned, as well as any closely linked genes that display synteny across plant species. The term “synteny” refers to the conservation of gene placement/order on chromosomes between different organisms. This means that two or more genetic loci, that may or may not be closely linked, are found on the same chromosome among different species. Another term for synteny is “genome colinearity”.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those set forth and described herein will be apparent to those skilled in the art from the foregoing description. Such modifications are also intended to fall within the scope of the appended claims.

The disclosure of each reference set forth herein is incorporated herein by reference in its entirety.

### Example 1

### Mapping of the Oryza sativa Goliath Locus

Identification of the chromosome comprising the Oryza sativa Goliath (GO) locus was performed using Cleaved Amplified Polymorphic Sequence markers (CAPS markers) and Simple Sequence Repeat markers (SSR markers). Located within the GO locus is the GO gene that, when mutated, confers an altered embryo phenotype in rice. DNA prepared from mutant rice plants showing an enlarged embryo phenotype was used as a source to identify the GO gene. According to Professor Yasuo Nagato (from the University of Tokyo, Tokyo, Japan) mutations in the GO gene, which cause the enlarged embryo phenotype, can only be propagated in heterozygote plants. This means that suppression mutations in the GO gene are lethal recessive. The sequences of two mutant alleles of the GO gene, go-1 and go-2, were identified in the present study.

Rice seeds from plants heterozygous for the go-1 mutation (Japonica rice cv. Taichung 65) were kindly provided by Professor Yasuo Nagato from the University of Tokyo, Tokyo, Japan.

Rice seeds from plants heterozygous for the go-2 mutation were obtained from a Japonica rice cv. Taichung 65 tissue culture population. Rice cells were incubated in tissue culture for 4 months to obtain a tissue culture population. It is known that tissue-culture frequently induces mutations that are transferred to the regenerated plants (see for example, Kaeppler et al. (2000) Plant Mol. Biol 43:179-188). 20,000 rice plants were regenerated from this tissue-culture population. Rice seeds having an enlarged embryo were retrieved and screened for a go mutation.

F1 seeds were obtained by crossing rice plants obtained from seeds heterozygous for the go-1 or the go-2 mutation (female parent), with plants from an Indica rice cultivar Kasalath (male parent). F1 plants obtained from these F1 seeds were selfed to obtain F2 plants that would produce F2 seeds. F2 seeds homozygous for the go-1 or go-2 mutation were used to prepare genomic DNA to identify the GO locus. These homozygous seeds will be referred to herein as go/go mutant seeds.

Genomic DNA was prepared from F2 seeds homozygous for the go-1 or the go-2 mutation obtained above (go/go mutant seeds). These F2 seeds were sterilized and put on MS media for callus induction. Between 100 and 500 mg of one-month-old callus tissue derived from single homozygous go/go mutant seeds were used for DNA extraction using DNAzol® buffer (Life Technologies Inc., Rockville, Md., 20849) following the manufacturers instructions.

Several CAPS markers and SSR markers were developed using allele-specific PCR primers designed based on rice genomic sequence information retrieved from GenBank®. This information has been released to GenBank® by the Rice Genome Project Group (RGP) (Harushima et al. (1998) Genetics 148:479-494). Additional SSR markers were designed based on BAC sequences released by the Clemson University Genomics Institute (CUGI) (Chen et al (2002) Plant Cell 14:537-545).

CAPS markers and SSR markers were amplified in 25 μL reactions containing 2.5 μL 2.5 mM dNTPs, 1.5 μL 25 mM MgCl₂, 25 ng genomic DNA extracted as above, 0.15 μL Amplitaq Gold® (Perkin Elmer) and 2.5 μL PCR buffer. Thermal cycle conditions were 10 minutes at 95° C. followed by 35 cycles of 45 seconds at 94° C., 45 seconds at 56° C., and 45 seconds at 72° C., after which the machine was set at 72° C. for 7 minutes.

SSR markers were analyzed by comparing the amplified DNA fragments obtained. CAPS markers were analyzed by digesting the amplified DNA fragment with a restriction endonuclease in a 15 μL digestion reaction containing 3 μL of amplified DNA, 1.5 μL 10× reaction buffer, and 0.5 μL enzyme (Promega, Madison, Wis.). The digestion reaction was incubated for 1 hour at 37° C. and polymorphisms were analyzed by loading the digests on a 2.5% agarose gel and separating by electrophoresis.

CAPS marker C3-145 and SSR marker SSR45 were designed based on two CUGI BAC clones covering approximately 10 cM of the rice genome as described below. The locus containing the GO gene was mapped to Chromosome 3 using genomic DNA prepared from homozygous go/go mutant seeds, CAPS marker C3-145 and SSR marker SSR45.

Marker SSR45 was amplified using oligonucleotide primers SSR 45F and SSR 45R. Oligonucleotide primers SSR 45F and SSR 45R were developed based on BAC end sequences of the CUGI clone OSJNBa0005B12 which is localized at about 155 cM of Chromosome 3. Oligonucleotide primers SSR 45F and SSR 45R are set forth in SEQ ID NO:1 and SEQ ID NO:2, respectively, and have the sequences shown as follows:

| | |
|---|---|
| 5′-CTCACGATCCTTACCTTGAATTG-3′ | SEQ ID NO: 1 |
| 5′-ATCCACTGTGTGCGTTTCTAGTT-3′ | SEQ ID NO: 2 |

This oligonucleotide primer set amplified a 203 by region flanking the tri-nucleotide repeat (AAG)₆₆ showing polymorphism between Indica and Japonica cultivars.

Marker C3-145 was amplified using oligonucleotide primers C3-145F and C3-145R. Oligonucleotide primers C3-145F and C3-145R were developed based on CUGI clone OSJNBa0091J19 which maps at 145.6 cM of chromosome 3. Oligonucleotide primers C3-145F and C3-145R are set forth in SEQ ID NO:3 and SEQ ID NO:4, respectively, and have the sequences shown as follows:

| | |
|---|---|
| 5′-ACGGGTTGTTTCACTTACAGGT-3′ | SEQ ID NO: 3 |
| 5′-TGTTTACCAAACTAGCCACCCAT-3′ | SEQ ID NO: 4 |

This oligonucleotide primer set amplified a 1128 by fragment. Digestion with the 4-cutter enzyme Hha I showed polymorphism between Indica and Japonica cultivars.

Eighty five seeds homozygous for either go-1 or go-2 were analyzed. One showed a recombination point with marker C3-145 and 6 showed recombination points with marker SSR45 indicating that the GO locus was closer to marker CAP C3-145 than to Marker SSR45.

Additional oligonucleotide primers were designed based on the precise contig information available at the CUGI web site. These oligonucleotide primers were used to obtain SSR markers 60J21 and 24J04.

SSR marker 60J21 was amplified using oligonucleotide primers b0060j21ssr1 and b0060j21ssr2. Oligonucleotide primers b0060j21ssr1 and b0060j21ssr2 were developed based on BAC sequences of CUGI clone OSJNBb0060J21. Oligonucleotide primers b0060j21ssr1 and b0060j21ssr2 are set forth in SEQ ID NO:5 and SEQ ID NO:6, respectively, and have the sequences shown as follows:

| | |
|---|---|
| 5′-GCCATCCTCCACTCCTCATC-3′ | SEQ ID NO: 5 |
| 5′-TATGCAAACTGGACGAATTACCC-3′ | SEQ ID NO: 6 |

Amplification using the oligonucleotide primers set forth in SEQ ID NO:5 and SEQ ID NO:6 resulted in a 211 by fragment. This fragment is located approximately at position 130 Kb of the clone flanking the compound di-nucleotide repeat (GA)₅ (GT)₃ (GA)₄ showing polymorphism between Indica and Japonica cultivars.

SSR marker 24J04 was amplified using oligonucleotide primers b0024J04ssr3 and b0024J04ssr4. Oligonucleotide primers b0024J04ssr3 and b0024J04ssr4 were developed based on BAC sequences of the CUGI clone OSJNBb0024J04. Oligonucleotide primers b0024J04ssr3 and b0024J04ssr4 are set forth in SEQ ID NO:7 and SEQ ID NO:8, respectively, and have the sequences shown as follows:

| | |
|---|---|
| 5′-ATAAGCAAGCTCACACACACCTC-3′ | SEQ ID NO: 7 |
| 5′-GCTAGCTACTCTCCACCACTCTG | SEQ ID NO: 8 |

Amplification using the oligonucleotide primers set forth in SEQ ID NO:7 and SEQ ID NO:8 resulted in a 214 by fragment. This fragment maps at a position approximately 37 Kb of clone OSJNBb0024J04 flanking the di-nucleotide repeat (CT)₁₅.

Thus, based on the CUGI physical map information, SSR markers 60J21 and 24J04 are located on the external 2 BACs of a contig comprised 3 different BACs. The internal BAC clone (OSJNBa0087o09) overlaps, on the right side, with about 28 Kb of BAC OSJNBb0024J04 and, on the left side, with about 5 Kb of BAC OSJNBb0060J21. A physical distance of about 160 Kb, encompassing the entire OSJNBa0087o09 clone separates the two markers.

Four recombination breakpoints, two from each side of the GO locus, were identified using SSR marker 24J04.

Oligonucleotide primers a87o09ssr1 and a87o09ssr2 were designed in the region around 62 Kb of clone OSJNBa0087o09. Oligonucleotide primers a87o09ssr1 and a87o09ssr2 are shown in SEQ ID NO:9 and SEQ ID NO:10, respectively, and have the sequences shown as follows:

| | |
|---|---|
| 5′-GATGTCCTCTCCCACCTTGC-3′ | SEQ ID NO: 9 |
| 5′-AGGGTGTACAGTCAGCACCTCTC-3′ | SEQ ID NO: 10 |

Amplification, using the primers shown in SEQ ID NO:9 and SEQ ID NO:10, produced a 123 by fragment comprising the di-nucleotide repeat (AG)₇. One recombination breakpoint was found when screening with this primer set the 4 recombination breakpoints identified using SSR marker 24J04. Thus, the GO locus lies between position 130 Kb of clone OSJNBb0060J21 and position 62 Kb of clone OSJNBa0087o09.

The sequence of BAC OSJNBa0087o09 was searched for the presence of open reading frames. Six regions were identified showing similarities to genes found in the GenBank database as well as the DuPont proprietary EST database. Two candidate genes were amplified from wild type, go-1, and go-2 genomic DNA and the sequences compared. No mutations were identified in one of the genes. The sequences of the two mutant alleles (go-1 and go-2) showed differences with the wild-type in the region comprising the rice gene homologous to the Arabidopsis thaliana glutamate carboxypeptidase (Amp1) found in the NCBI database as gi 15624091.

The nucleotide sequence of the genomic rice GO gene is shown in SEQ ID NO:11. The coding region of this genomic nucleotide sequence is divided into 10 exons corresponding to nucleotides 4152 through 5102, nucleotides 6002 through 6244, nucleotides 6530 through 6682, nucleotides 6828 through 6896, nucleotides 9134 through 9221, nucleotides 9600 through 9683, nucleotides 9947 through 10035, nucleotides 10386 through 10499, nucleotides 10966 through 11100, and nucleotides 11300 through 11524. Nucleotides 11522 through 11524 correspond to a stop codon. The amino acid sequence obtained by translating the above-mentioned exons is set forth in SEQ ID NO:12. The go-1 allele has an A instead of G at the first base of the first intron of the gene (nucleotide 5103 of SEQ ID NO:11) causing mis-splicing of the gene. The go-2 allele carries a 29 nucleotide deletion starting at position 4511 of the genomic sequence, which corresponds to nucleotide 458 of the coding region, causing a frameshift and introducing a premature stop codon after amino acid 282.

A cDNA clone encoding a rice GO was identified by searching the DuPont proprietary database using the amino acid sequence deduced from the rice GO gene (set forth in SEQ ID NO:12). The cDNA insert, SEQ ID NO:13, from clone rls6.pk0079.c3 encodes the amino acid sequence set forth in SEQ ID NO:14. Clone rls6.pk0079.c3 was obtained from a library prepared from Oryza sativa leaves of plants susceptible to infection with the fungal strain Magnaporthe grisea 4360-R-67 (AVR2-YAMO). The leaves were harvested 15 days after the plants germinated and 6 hours after infection with the fungus.

The nucleotide sequence of the cDNA insert from clone rls6.pk0079.c3 is set forth in SEQ ID NO:13. The amino acid sequence deduced from translating nucleotides 100 through 2247 from SEQ ID NO:13 is set forth in SEQ ID NO:14. Nucleotides 2248-2250 of SEQ ID NO:13 correspond to the stop codon. The first 8 nucleotides of SEQ ID NO:13 correspond to a linker used in the preparation of the library. The amino acid sequence set forth in SEQ ID NO:14 is identical to that set forth in SEQ ID NO:12.

### Example 2

### Identification of a Zea maize Ortholog of the Oryza sativa GO Gene

A Zea maize ortholog of the Oryza sativa GO gene was identified using two different approaches. The terms “maize” and “corn” are used interchangeably herein.

A lambda genomic DNA library was prepared using 20-day-old seedlings from maize inbred line B73. This library was screened using the Oryza sativa GO gene from Example 1. Screening of the genomic library led to the identification of a corn ortholog of the rice GO gene. The sequence of the corn ortholog of the GO gene was used to screen a Du Pont proprietary EST database. Screening of the Du Pont proprietary EST database led to the identification of clone csc1c.pk003.k10 as comprising a corn ortholog of the rice GO gene. Clone csc1c.pk003.k10 was obtained from a cDNA library prepared using 20-day-old seedlings from maize inbred line B73 which were germinated in the cold. The nucleotide sequence of the cDNA insert in clone csc1c.pk003.k10 encoded a partial ortholog of GO comprising nucleotides 568-2260 as set forth in SEQ ID NO:15.

A second approach involved screening a BAC genomic library with the Oryza sativa GO gene from Example 1: This approach led to the identification of clone BACM2.pk146.m06 as containing a Zea maize ortholog of the Oryza sativa GO gene. The nucleotide sequence of the insert in this BAC clone is set forth in SEQ ID NO:17. Comparison of the nucleotide sequence set forth in SEQ ID NO:17 with the nucleotide sequence of the cDNA insert in clone csc1c.pk003.k10 (set forth in SEQ ID NO:15) indicated that the coding sequences were nearly identical. Thus, there appears to be only one corn ortholog of the rice GO gene. The complete coding region for the corn GO ortholog is set forth in nucleotides 1 to 2139 of SEQ ID NO:15, which encodes a polypeptide having the amino acid sequence set forth in SEQ ID NO:16, with nucleotides 2140-2142 corresponding to a stop codon. The first 567 nucleotides of SEQ ID NO:15, corresponding to approximately half of exon 1, was obtained from BACM2.pk146.m06 (nucleotides 2019 to 2585 of SEQ ID NO:17.) The exons comprising the corn GO ortholog are found in SEQ ID NO:17 at nucleotides: 2019-2963, 4541-4783, 5187-5342, 5664-5729, 8094-8181, 8885-8968, 9158-9246, 9511-9624, 9781-9915, and 9990-10211, with nucleotides 10209-10211 corresponding to the stop codon.

The function of the corn GO ortholog was evaluated using a TUSC mutant population. The Trait Utility System for Corn (TUSC) is a method that employs genetic and molecular techniques to facilitate the study of gene function in corn (U.S. Pat. No. 5,962,764). TUSC mutant insertions in the corn GO ortholog were identified in DNA from F1 progeny plants as described in U.S. Pat. No. 5,962,764. F2 kernels from self fertilized F1 plants were obtained from a Pioneer HiBred proprietary TUSC mutant population. DNA obtained from these F2 kernels was used for genotyping. Kernels identified as homozygous for a mutator insertion in the corn GO ortholog gene were then analyzed phenotypically.

Two independent mutator insertions were retrieved and named “go 114 knockout” and “go 171 knockout”. Both of these insertions were detected in the first exon of the corn ortholog of the Oryza sativa GO gene. The insertion in the go 114 knockout was found to reside 100 nucleotides after the initiator ATG codon of the corn GO gene ortholog. The insertion in the go 171 knockout was found at nucleotide 533 of the open reading frame of the corn GO ortholog.

Genotyping of the go 114 knockout and go 171 knockout mutator insertions was carried out by amplifying genomic DNA from F2 kernels obtained from self-fertilized F1 plants originally identified as having a mutator insertion in the corn GO ortholog. Amplification conditions were the same as those set forth in Example 1.

DNA from corn plants having the go 171 knockout mutator insertion was genotyped using oligonucleotide primers 171 muF, 63289, and 9242mu tir. Oligonucleotide primers 171muF and 63289 were developed based on the nucleotide sequence of the corn ortholog of the GO gene. Oligonucleotide primer 9242mu tir is a degenerate primer designed to anneal only to a TUSC mutator element. Oligonucleotide primers 171muF, 63289, and 9242mu tir have the nucleotide sequences set forth in SEQ ID NO:18, SEQ ID NO:19, and SEQ ID NO:20, respectively, and have the sequences shown as follows:

SEQ ID NO: 18

5′-TGTTCGTCAACCTCGGCCGCGAGGAGG-3′

SEQ ID NO: 19

5′-AAACCGCTGCTTGACTGCCTTATCGTC-3′

SEQ ID NO: 20

5′-AGAGAAGCCAACGCCAWCGCCTCYATTTCGTC-3′

Genomic DNA was amplified from F2 kernels obtained from self-fertilizing F1 plants having the go 171 knockout mutator insertion.

Amplification using the oligonucleotide primers set forth in SEQ ID NO:18 and SEQ ID NO:19 was expected to produce a 293 bp fragment if at least one copy of the DNA did not have the mutator insertion in the GO homolog gene.

Amplification using the oligonucleotide primers set forth in SEQ ID NO:20 and SEQ ID NO:19 was expected to produce a 330 by fragment if at least one copy of the DNA had a mutator insertion in the GO homolog gene. Furthermore, if the 330 by fragment was found and not the 293 by fragment, then this would indicate that the plants were homozygous for the mutator insertion. However, if the 330 by fragment was found along with the 293 by fragment, then this would indicate that the plants were heterozygous for the mutator insertion.

Genotyping results of F2 kernels obtained from self-fertilized F1 corn plants having the go 171 knockout mutator insertion showed that, as expected, the mutator insertion segregated 3:1.

Some of the kernels analyzed produced a 293 by fragment when amplified using the primers set forth in SEQ ID NO:18 and SEQ ID NO:19. This result indicated that at least one copy of the DNA from some kernels did not possess the go 171 mutator insertion:

Some of the kernels analyzed produced a 330 by fragment when amplified using the primers set forth in SEQ ID NO: 20 and SEQ ID NO:19. This result indicated that at least one copy of the DNA from some kernels did possess the go 171 mutator insertion because a 330 by fragment was produced.

Some of the kernels produced both a 293 by fragment and a 330 by fragment.

Accordingly, genotyping results identified some corn kernels as homozygous for the go 171 mutator insertion. Amplification of DNA from these corn kernels produced only a 330 by fragment and not a 293 by fragment. Phenotypical analysis of corn kernels homozygous for the go 171 knockout mutator insertions is described below.

Genotyping also Identified some corn kernels as heterozygous for the go 171 mutator insertion. Amplification of DNA from these corn kernels produced both a 330 by fragment and a 293 by fragment.

Similarly, DNA from F2 kernels obtained from self-fertilizing F1 corn plants having the go 114 knockout mutator insertion was genotyped using oligonucleotide primers 63289, 9242mu tir, and 93288. Oligonucleotide primers 63289 and 9242mu tir are described above and have the sequences shown in SEQ ID NO:19 and SEQ ID NO:20, respectively. Oligonucleotide primer 63288 was developed based on the nucleotide sequence of the corn ortholog of the GO gene. Oligonucleotide primer 63288 has the nucleotide sequence set forth in SEQ ID NO:21:

| | |
|---|---|
| 5′-GAACCGGCTTGTGCGGTCAGTTC-3′ | SEQ ID NO: 21 |

Genomic DNA was amplified from F2 kernels obtained from self-fertilizing F1 plants having the go 114 knockout mutator insertion.

Amplification using the oligonucleotide primers set forth in SEQ ID NO:21 and SEQ ID NO:19 was expected to produce a 835 by fragment if at least one copy of the DNA did not have the mutator insertion in the GO homolog gene.

Amplification using the oligonucleotide primers set forth in SEQ ID NO:20 and SEQ ID NO:19 was expected to produce a 771 by fragment if at least one copy of the DNA had a mutator insertion in the GO homolog gene. Furthermore, if the 771 by fragment was found and not the 835 by fragment, then this would indicate that the plants were homozygous for the mutator insertion. However, if the 835 by fragment was found along with the 771 by fragment, then this would indicate that the plants were heterozygous for the mutator insertion.

Genotyping results of F2 kernels obtained from self-fertilized F1 corn plants having the go 114 knockout mutator insertion showed that, as expected, the mutator insertion segregated 3:1.

Some of the kernels analyzed produced a 771 by fragment when amplified using the primers set forth in SEQ ID NO:21 and SEQ ID NO:19. This result indicated that at least one copy of the DNA from some kernels did not possess the go 114 mutator insertion.

Some of the kernels analyzed produced a 835 by fragment when amplified using the primers set forth in SEQ ID NO: 20 and SEQ ID NO:19. This result indicated that at least one copy of the DNA from some kernels did possess the go 114 mutator insertion because a 835 by fragment was produced.

Accordingly, genotyping results identified corn kernels homozygous for the go 114 mutator insertion. Amplification of DNA from these corn kernels produced only a 771 by fragment and not a 835 by fragment. Phenotypical analysis of corn kernels homozygous for the go 114 knockout mutator insertions is described below.

Genotyping also identified some corn kernels as heterozygous for the go114 mutator insertion. Amplification of DNA from these corn kernels produced both a 771 by fragment and an 835 by fragment.

Kernels homozygous for a mutator insertion in the corn ortholog of the GO gene showed an embryo/endosperm phenotype comprising (a) lack of complete development, (b) lack of embryo axis and (c) possible increase of scutellar mass.

Kernels homozygous for the go 171 mutator insertion and the go 114 mutator insertion were planted. Kernels homozygous for a mutator insertion in the corn ortholog of the GO gene did not germinate when planted. The results obtained for corn were comparable to those obtained for rice, specifically, suppression of the corn GO ortholog gene is lethal recessive.

### Example 3

### Complementation of an Oryza sativa Go Mutant with the Oryza sativa GO Gene

Confirmation of the function of the Oryza sativa GO gene, identified in Example 1, was performed using genetic complementation: Rice callus cells derived from wild type and go/go mutant seeds were transformed with a genomic DNA fragment comprising the Oryza Sativa GO gene. Cloning of the genomic fragment comprising the wild type Oryza sativa GO gene and transformation into rice callus cells follows:

Transformation vector pML18 was derived from commercially available vector pGEM9z (obtained from Gibco-BRL which is owned by Invitrogen, Carlsbad, Calif.). Transformation vector pGEM9z was modified by inserting a Sal I fragment into the Sal I site.

This Sal I fragment comprised the following: (i) a cauliflower mosaic virus 35S promoter, driving expression of (ii) a bacterial hygromycin phosphotransferase open reading frame, (iii) followed by nucleotides 848 to 1550 of the 3′ end of the nopaline synthase gene. Insertion of this Sal I fragment produced transformation vector pML18. The bacterial hygromycin phosphotransferase gene confers resistance to hygromycin which is used as a selectable marker for rice transformation. The nucleotide sequence of transformation vector pML18 is set forth in SEQ ID NO:22.

A 12 Kb DNA fragment containing the wild type Oryza sativa GO gene was obtained by digesting BAC clone OSJNBa0087o09 with restriction endonucleases Spe I and Avr II. Transformation vector pML18 was digested with restriction endonuclease Spe I. The Spe I-digested transformation vector pML18 and the 12 Kb DNA fragment containing the wild type Oryza sativa GO gene were ligated to produce vector OsGOpML18.

Vector OsGOpML18 was introduced into rice callus cells derived from wild type rice seeds and from go/go mutant seeds using a Biolistic PDS-1000/He gun (BioRAD Laboratories, Hercules, Calif.) and the particle bombardment technique (Klein et al. (1987) Nature (London) 327:70-73).

Specifically, embryogenic callus cultures derived from the scutellum of go/go rice seeds were used as source material for transformation experiments. This material was generated by germinating sterile rice seeds on N6-2,4D media (N6 salts, N6 vitamins, 2.0 mg/l 2,4-D, 100 mg/L myo-inositol, 300 mg/L casamino acids, and 2.7 g/L proline) in the dark at 27-28° C. Embryogenic callus proliferating from the scutellum of the embryos was then transferred to fresh N6-2,4D media. Callus cultures were maintained by routine sub-culture at two-week intervals and used for transformation within 4 weeks of initiation.

Callus was prepared for transformation by arranging 0.5-1.0 mm callus pieces approximately 1 mm apart in a circular area of about 4 cm in diameter in the center of a circle of Whatman #541 paper placed on CM media and incubating in the dark at 27-28° C. for 3-5 days. Vector OsGOpML18 was introduced into rice callus cells from wild type or go/go seeds using a Biolistic PDS-1000/He gun (BioRAD Laboratories, Hercules, Calif.).

Mutant callus transformed with vector OsGOpML18 regenerated into plants confirming that the Oryza sativa GO gene is capable of complementing a go mutant phenotype. Unfortunately, all of the resulting plants were sterile, so it was impossible to evaluate the seeds for a go phenotype.

On the other hand, mutant callus transformed with vector pML18 did not regenerate into plants because this vector did not contain an Oryza sativa GO gene. Since suppression of the GO gene is a lethal recessive mutation, then seeds having an enlarged embryo phenotype and homozygous for a go mutation will not produce plants. Thus, callus obtained from the go/go seeds will not regenerate into plants.

### Example 4

### Complementation of an Arabidopsis thaliana Amp1 Mutant with the Oryza sativa GO Gene

As disclosed in Example 1 above, the Oryza sativa GO gene shares sequence similarity with the Arabidopsis thaliana Amp 1 gene. Thus, the ability of the Oryza sativa GO gene to complement an amp 1 mutant phenotype was studied. Arabidopsis thaliana amp 1 mutant seeds (stock No. CS8324) were obtained from the Arabidopsis Biological Resource Center (ABRC). Plants were grown and, using Agrobacterium tumefaciens, transformed with a binary vector comprising a rice GO gene.

A portion of the cDNA insert in clone rls6.pk0079.c3 (described in Example 1 above) was amplified using primer GO-xhoF1 and a 17-specific primer. Oligonucleotide primer GO-xhoF1 was designed based on the rice GO sequence and introduces an Xho I restriction endonuclease site in the region 5′ to the initiator ATG of the rice GO gene. The 17-specific primer was designed to anneal to the T7 terminator in the pBlueScript vector. Oligonucleotide primer GO-xhoF1 is shown in SEQ ID NO:23 and the 17-specific primer is shown in SEQ ID NO:24. These primers have the sequences shown:

| | |
|---|---|
| 5′-ATTAACTCGAGCGCTGCGCTGTG-3′ | SEQ ID NO: 23 |
| 5′-CGGGATATCACTCAGCATAATG-3′ | SEQ ID NO: 24 |

Amplification was performed under the same conditions as described in Example 1 above. Amplified DNA was digested with Xho I and inserted into Xho I-digested binary vector pBE851.

Binary vector pBE851 comprises a hygromycin resistance gene for transformation selection. This vector also comprises polynucleotides corresponding to a 35S promoter and a phaseolin terminator region separated by an Xho I site. The resultant binary vector, OsGOBE851, comprises a 35S promoter operably linked to the Oryza sativa GO open reading frame, followed by the phaseolin terminator region. The nucleotide sequence of binary vector OsGOBE851 is set forth in SEQ ID NO:25.

Binary vector OsGOBE851 was transformed into Agrobacterium tumefaciens strain C58, grown in LB at 25° C. to OD600˜1.0. Cells were then pelleted by centrifugation and resuspended in an equal volume of 5% sucrose/0.05% Silwet L-77 (OSI Specialties, Inc). At early bolting, soil-grown amp 1 mutant Arabidopsis thaliana plants grown from stock No. CS8324 were top watered with the Agrobacterium suspension. A week later, the same plants were top watered again with the same Agrobacterium strain in sucrose/Silwet. The plants were then allowed to set seed as normal. The resulting T₁ seed were sown on soil, and transgenic seedlings were selected by spraying with glufosinate (Finale®; AgrEvo; Bayer Environmental Science).

Genomic DNA from wild-type-looking transgenic plants was amplified using primers GO 1566F and GO 1747R. Primers GO 1566F and GO 1747R were developed based on the rice GO gene sequence. Oligonucleotide primers GO 1566F and GO 1747R are shown in SEQ ID NO:26 and SEQ ID NO:27, respectively, and have the sequences shown:

| | |
|---|---|
| 5′-GATGGAAAAGCATGGTGATCCAC-3′ | SEQ ID NO: 26 |
| 5′-GAACCCATTTGCTATTTTCCATC-5′ | SEQ ID NO: 27 |

Amplified DNA showed that the rice GO gene from binary vector OSGOBE851 was present in the transgenic plants having a wild-type appearance.

Genomic DNA from wild-type-looking transgenic plants was also amplified using primers Amp1-1566F and Amp1-1747R. Oligonucleotide primers Amp1-1566F and Amp1-1747R were designed based on the AMP1 sequence found in the NCBI database as gi 15624091. Oligonucleotide primers Amp1-1566F and Amp1-1747R are shown in SEQ ID NO:28 and SEQ ID NO:29, respectively, and have the sequences shown:

| | |
|---|---|
| 5′-GATGATCCACAACGCAGATCCAT-3′ | SEQ ID NO: 28 |
| 5′-TAACAGAGACTTTCCCTTCTAAG-3′ | SEQ ID NO: 29 |

Sequencing of the amplified DNA confirmed that the transgenic plants having a wild-type appearance, indeed, had the amp 1 mutation. The amp1 mutation has been described as a change of G to A in the Amp1 exon 7 (Helliwell et al, 2000). Two transgenic plants having a wild-type appearance had both, the rice GO gene and the amp1 mutation. Thus, the rice GO gene is capable of complementing an amp1 mutant phenotype. These results confirm that the rice GO gene has the same function as the Amp/gene.

What is claimed is:

1. An isolated polynucleotide comprising:
(a) a nucleic acid sequence encoding a polypeptide involved in altering embryo/endosperm size during seed development, said polypeptide having at least 80% amino acid sequence identity, based on the Clustal V method of alignment, when compared to an amino acid sequence selected from the group consisting of SEQ ID NOs:12 and 16; or
(b) the nucleic acid sequence set forth in SEQ ID NO:11 wherein said sequence comprises at least one of the following modifications:
(i) nucleotide 5103 is a T residue instead of a C; or
(ii) nucleotides 4511 through 4540 have been deleted; or
(c) the nucleic acid sequence set forth in SEQ ID NO:11, 13; 15, or 17; or
(d) all or part of the isolated polynucleotide comprising sequences of (a), (b), or (c) for use in suppression of endogenous nucleic acid sequences encoding polypeptides involved in altering embryo/endosperm size during seed development; or
(e) the full complement of (a), (b), (c); or (d).

2. The isolated polynucleotide of claim 1 wherein the amino acid sequence identity is at least 85%.

3. The isolated polynucleotide of claim 1 wherein the amino acid sequence identity is at least 90%.

4. The isolated polynucleotide of claim 1 wherein the amino acid sequence identity is at least 95%.

5. The isolated polynucleotide of claim 1 wherein the amino acid sequence identity is 100%.

6. An isolated polynucleotide encoding a encoding a polypeptide involved in altering embryo/endosperm size during seed development wherein said isolated polynucleotide hybridizes under stringent conditions to one of the nucleotide sequences set forth in SEQ ID NOs:11, 13; 15, and 17.

7. An isolated polynucleotide comprising a nucleotide sequence encoding a polypeptide involved in altering embryo/endosperm size during seed development, wherein the nucleotide sequence has at least 80% sequence identity, based on the BLASTN method of alignment, when compared to a nucleotide sequence as set forth in SEQ ID NOs:11, 13; 15, and 17.

8. A recombinant DNA construct comprising the isolated polynucleotide of any one of claims 1 through 7 operably linked to at least one regulatory sequence.

9. A plant comprising in its genome the recombinant DNA construct of claim 8.

10. Seeds obtained from the plant of claim 9.

11. Oil obtained from the seeds of claim 10.

12. The plant of claim 8 wherein said plant is selected from the group consisting of rice, corn, sorghum, millet, rye, soybean, canola, wheat, barley, oat, beans, and nuts.

13. Transformed plant tissue or plant cells comprising the recombinant DNA construct of claim 8.

14. The transformed plant tissue or plant cells of claim 13 wherein the plant is selected from the group consisting of rice, corn, sorghum, millet, rye, soybean, canola, wheat, barley, oat, beans, and nuts.

15. A method of altering embryo/endosperm size during seed development in a plant comprising:
(a) transforming plant cells or plant tissue with the recombinant DNA construct of claim 8;
(b) regenerating transgenic plants from the transformed plant cells or plant tissue of (a);
(c) screening the transgenic plants of (b) for seeds having an altered embryo/endosperm size based on a comparison of embryo/endosperm size of seeds obtained from non-transformed plants.

16. The method of claim 15 wherein said plant is selected from the group consisting of rice, corn, sorghum, millet, rye, soybean, canola, wheat, barley, oat, beans, and nuts.

17. A method of mapping genetic variations related to controlling embryo/endosperm size and/or altering oil phenotype in plants comprising:
(a) crossing two plant varieties; and
(b) evaluating genetic variations with respect to
(i) a nucleic acid sequence selected from the group consisting of SEQ ID NOs:11, 13; 15, and 17; or
(ii) a nucleic acid sequence encoding a polypeptide selected from the group consisting of SEQ ID NOs:12 and 16;
in progeny plants resulting from the cross of step (a) wherein the evaluation is made using a method selected from the group consisting of RFLP analysis, SNP analysis, and PCR-based analysis.

18. The method of claim 17 wherein the plant is selected from the group consisting of rice, corn, sorghum, millet, rye, soybean, canola, wheat, barley, oat, beans, and nuts.

19. A method of molecular breeding to control embryo/endosperm size and/or altering oil phenotype in plants comprising:
(a) crossing two plant varieties; and
(b) evaluating genetic variations with respect to
(i) a nucleic acid sequence selected from the group consisting of SEQ ID NOs:11, 13, 15, and 17; or
(ii) a nucleic acid sequence encoding a polypeptide selected from the group consisting of SEQ ID NOs:12 and 16;
in progeny plants resulting from the cross of step (a) wherein the evaluation is made using a method selected from the group consisting of RFLP analysis, SNP analysis, and PCR-based analysis.

20. The plant of claim 19 wherein the plant is selected from the group consisting of rice, corn, sorghum, millet, rye, soybean, canola, wheat, barley, oat, beans, and nuts.
